(19) United States
(12) Patent Application Publication
(10) Pub. No.: US 2015/0093440 A1
(43) Pub. Date: Apr. 2, 2015

(54) AGGREGATE NANOPARTICULATE MEDICAMENT FORMULATIONS, MANUFACTURE AND USE THEREOF

(71) Applicants: Michiel M. Van Oort, Research Triangle Park, NC (US); John N. Hong, Research Triangle Park, NC (US)

(72) Inventors: Michiel M. Van Oort, Research Triangle Park, NC (US); John N. Hong, Research Triangle Park, NC (US)

(73) Assignee: GLAXO GROUP LIMITED, Brentford, Middlesex (GB)

(21) Appl. No.: 13/879,103

(22) PCT Filed: Oct. 13, 2011
(86) PCT No.: PCT/US2011/056166

§ 371 Date: May 5, 2014

Related U.S. Application Data
(60) Provisional application No. 61/393,586, filed on Oct. 15, 2010.

(51) Int. Cl. A61K 31/56; A61K 9/16; A61K 31/4709

(52) U.S. Cl. CPC A61K 31/56; A61K 31/4709; A61K 9/1688; A61K 9/1623; A61K 9/1617
USPC 424/489; 514/312; 514/180; 514/784; 514/777

(57) ABSTRACT

A method of making aggregate particles suitable for a powder aerosol composition that includes forming a dispersion of nanoparticulate drug and/or excipient in a non-aqueous liquid, and spray-drying the dispersion to generate aggregate particles having a mass median aerodynamic diameter of less than or equal to about 100 microns, and particles generated by such method, and compositions of said particles.

### FIELD OF THE INVENTION

The following invention relates to powder compositions suitable for inhalation that contain aggregates comprising nanoparticulate drug particles and/or nanoparticulate excipient particles, and optionally a binder. The invention also relates to processes of producing such particles, and methods using such particles and particle compositions.

### BACKGROUND OF THE INVENTION

Inhaled medicines are delivered via the mouth or nose of a patient, for deposition in the pulmonary system. The pulmonary system includes the nasal mucosa, the throat and lungs. Target sites for therapy via inhalation are for example, the mucosal region of the nose, the oropharynx region of the throat, and the bronchiole smooth muscle region in the lung, and the alveolar region of the deep lung. Generally systemic delivery is achieved through deposition to the alveolar region of the lung or the mucosal area or the nose. Topical therapies are delivered to the nasal mucosa and the smooth muscle areas of the lungs.

Drugs delivered via the pulmonary route may be liquids or solids. The liquid or solid particles deposit in the pulmonary system based upon their aerodynamic size. For example, a particle or droplet larger than approximately 10 microns tend to deposit in the upper regions of the pulmonary system, such as the throat/larynx, first bifurcation of the lung. Particles having an aerodynamic size between 2 and 10 microns, such as larger than 3 microns up to 10 microns, such as from 3 to 6 microns, more particularly 4 to 5 microns, tend to settle in the smooth muscle areas of the bronchial region of the lungs. Generally, particles from 1 to 3 microns, and more particularly from 1 to less than 3 microns, such as about 2 microns in aerodynamic size, tend to settle in the alveolar region. It is appreciated that particles deposit in the pulmonary system based upon their aerodynamic size.

The aerodynamic behavior of inhaled particles is generally considered to be dependent on factors including the size, shape, and density of the particles making up an inhalable composition. Moreover, aerodynamic behavior and deposition is influenced by airflow characteristics, such as the air flow rate, and also delivery device characteristics, such as the pressure drop associated with particle aerosolization. Chemical and/or physical makeup of the inhaled particles and composition, if unstable, may change over time. For example, a change in the chemical composition of the active may result in a reduction in the quantity of active agent in the composition. A change in the crystalline form of the active agent may result in changes in bioavailability. Instability in the physical characteristics of a composition, for example due to particle growth, may lead to a lowering of the percentage of the particles in the composition having the desired aerodynamic size.

Developers of inhaled medicine must therefore be concerned with making particle compositions of particles of the desired aerodynamic size, which may be reproducibly delivered to a desired location in the pulmonary system. These compositions should be physically and chemically stable, such that their chemical and physical nature remains predictable and relatively constant during storage and their performance remains acceptable during the lifetime of the product. It is desirable that the compositions are capable of being manufacture in a controlled manner, and that this process is cost effective.

The present invention relates to aggregate particles for use in inhaled pharmaceuticals, as well as methods of making such aggregate particles. These may be delivered by a suitable inhalation delivery system, such as a pressurized metered dose inhaler (MDI) or from a dry powder inhaler (DPI).

Conventional powders used in DPIs and suspension based MDIs typically contain an active pharmaceutical agent that has been milled to a desired aerodynamic size. In a DPI, the active agent is generally admixed with a coarse carrier/diluent, such as lactose. Other additive materials may be presented to act as physical or chemical stabilizers, dispersants, taste masking agents, etc. In a suspension based MDI, the active agent is suspended in a low-boiling point liquid propellant. The propellant formulation may also include other materials which improve product performance, such as surfactants, etc.

There is a constant effort to improve upon the performance of existing inhalation delivery systems, including the performance of the compositions used in those systems. For example, the desire to improve the current particle based system to provide powders that can be effectively aerosolized to maintain a uniform dose and which can be easily separated from the carrier materials, so as to generate particles of a desired size for targeted site delivery in the pulmonary system, has in recent years, led to a considerable effort to engineer better inhaled particles. One goal of these efforts is the manufacture of particles which are chemically and physically more stable, have greater dispersion, aerosolization and cost efficiencies, so as to optimize inhalation aerosolization and delivery performance.

In such efforts, adhesion and detachment forces have been looked at as important factors determining a successful delivery of powdered drug into the lungs by inhalation. The adhesion forces which include Van der Waals, capillary, Coulomb and electrical (double layers) forces, influence powder flowability (and thus dose repeatability), aerosolization of the powders and particle deagglomeration during delivery. The adhesion and detachment behavior of particles is dependent on particle size, shape, surface factors, electric charge and hygroscopicity.

Previous efforts have involved, for example, improving the hydrophobic surface properties of particles to reduce susceptibility to capillary adhesion, looking at particle surface roughness in an effort to increase the effective separation distance between particles, looking at curved particle surface to reduce the contact areas, attempting to control particle surface and interior charge to reduce Coloumb and electrical forces, and studying an appropriate aerodynamic size to allow deposition at a selected site. Further focus has been directed at physical or chemical stability issues which lead to decreased product performance upon storage, such as decreases in dose uniformity over product life.

As mentioned above, powder blend formulations of micronized drug were pursued to these ends. The conventional approach to creating powder, suitable for MDIs and DPIs has been precipitation or crystallization of active compounds from a solution, followed by drying and milling to produce micronized drug particles. This milling size reduction process is a high energy generating process during which the drug particles may become highly charged and increasing their cohesiveness to each other. The milling process may also introduce surface and crystallographic damage, which raises concerns on the powder's stability and often results in particles with irregular fragments that could form strong aggregates. Additionally, the milling process may generate flat faceted surfaces that contain many corner sites for condensation to occur, thus increasing adhesion forces and leading to inefficient drug-particle break-up. Lastly, the multi-step processing causes a significant loss of materials during production as well as variability of product properties generated from different batches.

These issues may become apparent even after the micronized drug is formulated for MDIs by suspending the drug particles in a suitable propellant formulation, or formulating for a DPI after they are blended with suitable micronized carrier/diluent particles.

One alternative approach to size reduction by milling is spray-drying, which has been investigated to some success. Spray drying is a one-step continuous process which can directly produce particles of a desired size range. This approach is amenable to the production drug powders for inhalation delivery, see, e.g. U.S. Pat. No. 4,590,206, Broadhead, J., et al, “Spray Drying of Pharmaceuticals”, Drug Development and Industrial Pharmacy, 18(11&12), 1169-1206 (1992), M. Sacchetti, M. Van Oort, Spray Drying and Supercritical Fluid Particle Generation Techniques, “Inhalation Aerosols: Physical and Biological Basis for Therapy”, Marcel Dekker, 1996, and patent publications WO 96/32149, WO 97/41833, WO 97/44013, WO 98/31346 and WO 99/16419.

Spray drying generally involves a liquid atomizer and a particle collection system. The atomizer of the spray-drying process converts the liquid feed into dried particles by atomizing the feed to a spray form in a hot gaseous medium. Rapid evaporation of the droplets forms dried, solid particles, which can be separated from the gas using a cyclone, an electrostatic precipitator, or a filter. The method is capable of controlling the particle size and size distribution, particle shape and particle density, by manipulating the process conditions.

Particles may be generated from solutions or suspensions. WO 96/09814 describes, for example, the spray drying of budesonide and lactose in ethanol, Published PCT application WO 2001/49263, U.S. Pat. No. 6,001,336, U.S. Pat. No. 5,976,574 (hydrophobic drugs from organic suspensions), and U.S. Pat. No. 7,267,813 (crystalline inhalable particles comprising a combination of two or more pharmaceutically active compounds) also describe spray dried particles.

While spray drying is suitable for producing respirable sized particles, solid state properties (particularly crystallinity) may or may not be properly controlled. While spray drying from solutions can result in crystalline materials, as shown, for example, in Kumon, M. et al, “Can low-dose combination products for inhalation be formulated in single crystalline particles” Eur. J. Pharm. Sci, 40, 16-24 (2010), where combination particles of corticosteroid, long acting beta agonist and the sugar alcohol, mannitol, were co-spray dried from a solution to prepare composite crystalline particles to reduce the risk of amorphous derived instability and hygroscopicity, crystallinity is dictated by the kinetics of the spray-drying process and compound properties. The spray drying process, depending on whether solutions or suspensions are being sprayed, and the conditions under which the process occurs, may produce amorphous particles. Such amorphous spray dried particles may have physical and/or chemical stability problems and have an increased tendency to be hygroscopic, all of which are undesirable for pharmaceutical agents. Spray drying solutions having therapeutically active materials with or without excipients therein may produce amorphous material due to the rapid precipitation within the atomized droplets. Moreover, while crystalline materials may be produced, the resulting crystalline product may be of a kinetically preferred form, as opposed to the more thermodynamically stable form. Therefore, an undesirable polymorphic form may result. Further improvement in this area is desirable.

Obtaining crystalline materials reproducibly by spraydrying is further complicated when multiple materials are being used, while one of the components may crystallize as desired, another in the same particle may not.

Thus, while spray drying is suitable for producing respirable sized particles, solid state properties (particularly crystallinity) are not easily controlled. As mentioned, conventional spray drying involves introducing liquid droplets into a heated gas in a chamber, causing the liquid solvent to evaporate. This heat exchange occurs very rapidly, and transition from liquid to solid phase may be so rapid that the crystallization process is very difficult to control. Amorphous particles typically have physical and/or chemical stability problems and have a high tendency to be hygroscopic, all of which are undesirable for pharmaceutical agents.

To assist in crystalline particle generation via spray drying, investigators have turned to drying and collection processes under highly regulated conditions, see, for example, in EP1322301B1a heated flow tube reactor is employed to control the drying and crystal formation of spray dried of drug in solution. These approaches are however more costly, require specialized drying collection apparatuses, and may not be suitable for commercial scale production. As would be appreciated, avoiding adding complex manufacturing processes to such particle generation systems is preferred.

In recent years, attention has turned to nanoparticle drug delivery. Nanoparticles may afford certain advantages in inhaled therapies, particularly their increased rate of dissolution, which is desirable in cases where a pharmaceutically active ingredient is poorly soluble in the environment experienced in the respiratory tract, or where rapid release is desired. Nanoparticles, due to their very small size, tend to dissolve rapidly, thus they have been employed for very hydrophobic materials to assist in more rapid dissolution, or where a rapid onset of action is required, such as with immediate release medications.

Pharmaceutically active materials may be delivered as nanoparticles alone, or as nanoparticle components incorporated into larger composite particles which act as delivery vehicles. For example, US 2003-0166509 describes spray drying of nanoparticles to form respirable larger sized particles. The nanoparticles are entrapped in a skeletal framework of precipitated excipient which makes up a larger particle of respirable size. The respirable particles are described as achieving a “sustained action” of drug upon delivery to a target site in the lung, as these composite particles degrade more slowly than a bare nanoparticle and release material in the entrapped nanoparticles as this degradation occurs. Generally, nanoparticles are spray dried from an aqueous suspension. In order to assure the homogeneity of the suspension feed stock, these processes typically include a surfactant in the liquid phase. Spray-drying of nanoparticles from non-aqueous liquid media is also described in the literature. For example, U.S. Pat. No. 7,521,068 describes a process where materials are milled in non-aqueous media in the presence of a surface modifier, to produce nanoparticle compositions of spherically shaped aggregates of drug and surface modifier particles. The use of surfactants, although frequently used, may increase the risk of negative clinical side effects. Thus, removing the surfactant after particle production may be necessary, which increases costs or complexity in manufacturing, if such removal is possible. In spite of this, nanoparticles may manufactured to be essentially crystalline, which could also avoid the instability and hygroscopicity issues generally found in amorphous particles.

The present invention builds upon this background, and employing spray drying, permits control and efficiency in generating improved particles and particle compositions containing nanoparticles. In particular, it is a goal to provide one or more of the following benefits: increased control in the physical and/or chemical properties on inhaled compositions, particularly crystallinity; increased manufacturing and/or delivery efficiency; greater flexibility in manufacturing, which allows use of a single platform of technology over a variety of pharmaceutically active materials and excipients; an improved drug delivery profile; longer shelf life; providing increased choice to formulators, healthcare providers and/or patients.

### BRIEF DESCRIPTION OF FIGURES

FIG. 1 is a graphical depiction of a typical suspension particle size distribution results for a two-component co-milled suspension of drug and excipient, compared to milled drug alone.

FIG. 2 is a series of typical scanning electron micrographs of aggregate particles of the present invention. Sample 1 depicts a pure drug aggregate, Samples 2 and 3 depict two-component particle formulations comprised of nanoparticulate drug particles and nanoparticulate excipient particles.

FIG. 3 shows typical XRPD patterns for the input API-A, lactose monohydrate and L-leucine prior to organic bead milling.

FIG. 4A is a typical X-ray powder diffraction (XRPD) pattern of a two-component nanoparticulate liquid dispersion following organic bead milling. The nanoparticulate liquid dispersion input for Sample 2 comprised of 50:50 API-A:Lactose in Ethyl Acetate is displayed.

FIG. 4B is a typical XRPD pattern of a two-component powder. Sample 2 comprised of 50:50 API-A:lactose is displayed.

FIG. 5. is a series of typical scanning electron micrographs of two-component aggregate particles comprising nanoparticulate drug particles and a binder.

FIG. 6. is a series of typical scanning electron micrographs of three-component particle aggregate formulations comprised of nanoparticulate drug particles and nanoparticulate excipient particles and a binder.

FIG. 7 is a pair of scanning electron micrographs of three-component particle formulations comprised of nanoparticulate drug particles and nanoparticulate excipient particles, comprising two different excipient materials.

FIG. 8 is a depiction of a typical wet particle size distribution results for a three-component co-milled suspension consisting of nanoparticulate drug particles and nanoparticulate excipient particles, comprising two different excipient materials. The results for a suspension consisting of 45:45:10 API-A:lactose:leucine is depicted.

FIG. 9A is a typical XRPD pattern of a three-component nanoparticulate liquid dispersion following organic bead milling. The nanoparticulate liquid dispersion input for Sample 18 comprised of 45:45:10 API-A:Lactose:Leucine in Ethyl Acetate is displayed. Nanoparticulate liquid dispersion was allowed to dry for XRPD analysis.

FIG. 9B is a typical XRPD pattern of a three-component aggregate formulation (nanoparticulate drug particles and nanoparticulate excipient particles, comprising two different excipient materials) after spray drying The results for a suspension consisting of 45:45:10 API-A:lactose:leucine is depicted.

### SUMMARY OF THE INVENTION

The present invention broadly relates to a method of making aggregate particles suitable for a powder aerosol composition comprising:
   (a) forming a dispersion of nanoparticulate drug particles and/or nanoparticulate excipient particles in a non-aqueous liquid,
      wherein said drug particles and/or said excipient particles have a solubility of less than 10 mg/ml in said liquid dispersing media, and
      wherein the nanoparticulate drug particles have a preselected crystalline form,
      and wherein, when the nanoparticles dispersed in said dispersion do not comprise excipient, the non-aqueous liquid has no suspension homogenizing surfactant dissolved therein;
   (b) spray-drying the dispersion of nanoparticulate drug particles and/or nanoparticulate excipient particles to generate aggregate particles comprising nanoparticulate drug particles and/or nanoparticulate excipient particles,
      wherein the drug and/or excipient nanoparticles have maintained their preselected crystalline form, and
      wherein the aggregate particles have a mass median aerodynamic diameter of less than or equal to about 100 microns
      and wherein when the nanoparticles dispersed in said dispersion do not comprise excipient, the aggregate particles is substantially free of a homogenizing surfactant.

In a further embodiment, the present invention relates to a method of making a dry powder aerosol composition comprising:
(a) forming, in a non-aqueous liquid, a dispersion of nanoparticulate drug particles and/or nanoparticulate excipient particles, wherein said drug particles and/or excipient particles have a solubility of less than 10 mg/ml in said liquid dispersing media,
(b) spray-drying the dispersion of nanoparticulate drug particles and/or nanoparticulate excipient particles to form a dry powder of aggregates of the nanoparticulate drug particles and nanoparticulate excipient particles, wherein the aggregates comprise both drug and excipient particles and have the aggregates have an mass median aerodynamic diameter of less than or equal to about 100 microns.

The invention also relates to aggregates produced by these processes/methods, compositions containing such aggregate particles, and therapies for the treatment of diseases and conditions using such aggregate particles or compositions.

For example, a further embodiment of the invention relates to a composition comprising aggregate particles for use in an aerosol drug delivery system, wherein the aggregate particles comprise (a) nanoparticulate drug particles and/or (b) nanoparticulate excipient particles, and, optionally (c) binder, wherein the nanoparticulate drug and/or excipient particles have a pre-selected crystalline form.

The aggregate particles may be delivered to the patient in dosage forms suitable for inhalation through a metered dose inhaler (MDI) or dry powder inhaler (DPI). The aggregates may be delivered to a patient with or without additional excipient-only diluent or carrier particles.

The above method(s) allow for the production of aggregate particles which are of a size suitable for pulmonary delivery through the nose or mouth of a patient. The aggregate particles, however, are constructed of nanoparticles of drug and/or nanoparticles of excipient. The use of nanoparticles has been found to afford a number of potentially significant advantages in the areas of physical stability, and product performance.

The nanoparticulate drug and nanoparticulate excipient particles suitably have an effective average particle size less than 1000 nm, for example, they suitably have an effective average particle size less than about 400 nm, or suitably less than about 300 nm, or suitably less than about 250 nm, or suitably less than about 100 nm, or suitably less than about 50 nm.

In one preferred embodiment, the nanoparticulate drug and/or excipient particles have an effective average particle size of less than about 300 nm. In another preferred embodiment, the nanoparticulate drug and/or excipient particles have an effective average particle size of less than about 250 nm. In still further embodiments, the nanoparticulate drug and/or excipient particles have an effective average particle size of less than about 100 nm. In still further alternative embodiments, the nanoparticulate drug and/or excipient particles have an effective average particle size of less than about 50 nm.

Preferably, 50% or more of the nanoparticulate drug particles, and 50% or more of the nanoparticulate excipient particles have an average particle size of less than 1000 nm.

For example, the nanoparticulate drug particles may have an effective average particle size of less than about 400 nm.

Alternatively, the nanoparticulate excipient particles have an effective average particle size of less than about 400 nm.

Still further, the nanoparticulate drug particles and the nanoparticulate excipient particles may both have an effective average particle size of less than about 400 nm.

In certain embodiments, at least 70% of the drug and excipient nanoparticles in the aggregate particles have a particle size of less than about 1000 nm, for example, suitably, at least 90% of the drug and excipient nanoparticles have a particle size of less than about 1000 nm.

The methods described herein advantageously allow the nanoparticles making up the agglomerates to assure that a preselected crystalline form present prior to aggregate formation is maintained and is present in the final aggregates formed via the spray drying process. This ability to maintain the crystalline form, in some cases, allows the selection of a thermodynamically stable crystalline form to be used, where such form may not be assured if another particular spray drying and collection approach was employed, such as by solution based spray drying, where the drug and/or excipient was substantially dissolved in a given liquid phase.

The pre-selection and maintenance of the crystalline form of nanoparticulate drug and nanoparticulate excipient particles throughout the process reduces the risk of conversion of the physical makeup of the aggregates after aggregate manufacture, such as upon storage. This added control has benefits in meeting strict quality control requirements of national drug regulators, and the substantially crystalline product lends itself to longer shelf life.

The nanoparticulate drug and nanoparticulate excipient particles also lend themselves to the production of morphologically preferable aggregate constructs. The aggregates have very good dispersibility and improved fine particle fractions compared to micronized drug particles admixed with a coarse carrier. Moreover, the incorporation of nanoparticulate excipient is itself advantageous, as it allows for dose ranging studies to be conducted, as the concentration may be modified in determining an optimal dose, and at least in situations where the nanoparticulate excipient makes up the bulk of a given agglomerate, the particle-to-particle adhesion properties, and the aerosolization properties of the aggregate particles will be governed by the properties of excipient.

Still further, the process may avoid the necessity of employing a homogenizing surfactant in the non-aqueous liquid that the nanoparticle are suspended in prior to spray drying, which translates into the ability to directly spray dry particles without a surfactant which would end up as a residue in the aggregate particles produced.

In certain embodiments, the aggregate particles are formed from spray drying a non-aqueous dispersion of nanoparticulate drug particles, and nanoparticulate excipient particles, wherein the nanoparticulate drug particles and nanoparticulate excipient particles have a solubility in the non-aqueous liquid of less than about 10 mg/ml.

A further potential advantage to this methodology is that the preselected crystalline form of the nanoparticles of drug and nanoparticles of excipient act as a seed crystals. Thus, in the instance where a slight portion of the drug and/or excipient material is dissolved in the non-aqueous suspending media, the seed crystals may act as lattice templates that may “steer” the crystallization process toward formation of the preselected crystalline form when the liquid phase of aerosolized droplet is evaporating during the spray drying process.

In certain embodiments of the methods of the present invention, the method may also include inclusion of a binder. The binder is dissolved in the non-aqueous liquid phase of the dispersion. Such method includes the step of including a binder in the nanoparticulate non-aqueous dispersion prior to spray-drying. Following spray-drying, essentially every aggregate contains one or more nanoparticulate drug particle, one or more nanoparticulate excipient nanoparticle and binder.

Suitably, in certain embodiments, the binder is dissolved in the liquid phase of the non-aqueous dispersion, to facilitate formation of aggregates comprising nanoparticulate drug particles and nanoparticulate excipient particles upon spray drying. The binder may be a portion of the drug or excipient which has dissolved in the non-aqueous media, or may be separately added to the non-aqueous media.

In a further aspect of the method, the method of making aggregate particles further comprises a step of forming said nanoparticulate drug particles and/or nanoparticulate excipient particles, wherein said forming step comprises bead milling larger particles of said drug and/or said excipient in a non-aqueous liquid substantially in the absence of a homogenizing surfactant to generate nanoparticulate drug particles and/or nanoparticulate excipient particles.

The drug and excipient particles used to produce nanoparticulate particles may be bead milled together, simultaneously in the bead mill. Alternatively, the drug and excipient may be bead milled separately and dispersions containing the different types nanoparticulate nanoparticles may be combined/admixed prior to drying to form aggregates of the nanoparticulate drug particles and nanoparticulate excipient particles.

Suitably, in certain embodiments, the bead milling of the drug and/or the excipient is conducted in the absence of an intentionally added homogenizing surfactant in the non-aqueous dispersing media used in the bead milling process. By careful selection of non-aqueous liquid non-solvent, use of surfactant in the suspension undergoing bead milling may be avoided (e.g., the non-aqueous liquid non-solvent is sufficiently wetting to the drug and/or excipient nanoparticles, that homogeneity of the milling material is maintained). This iteration of the invention provides a significant advantage in eliminating unnecessary additives to the intermediate product that may have to be removed later in the manufacturing process, and avoids the potential that residues of a homogenizing surfactant would be present in the aggregates. Such surfactant may pose a possible toxicological issue, thus requiring it to be removed, for example by washing. Removing the surfactant may be difficult, as residual surfactant may remain even after this washing/extraction process.

In a further embodiment, the invention relates to a product by the process herein described, as well as pharmaceutical compositions comprising such product, and methods or treatment involving administration of such product and/or formulations thereof to an individual in need thereof.

In one embodiment, the composition including the aggregate particles is formed by blending said aggregate particles with carrier or diluent particles which comprise excipient material, for example, lactose or mannitol, optionally with a further agent, such as lubricant, for example, magnesium stearate or calcium stearate. Various drugs, excipients and binders are discussed further below.

In one embodiment, the excipient may be lactose in a milled or micronized form. In such embodiment, the composition comprises drug containing aggregates admixed with lactose. It is believed that such formulation may possess beneficially enhanced delivery and dispersion efficiencies. This approach also may be advantageously used to further dilute high potency drugs, in instances where further diluents is desirable to allow for metering, and dose adjustment.

Thus, a further aspect of the present invention is a pharmaceutical formulation/composition of a dry powder aerosol composition for use in a dry powder inhaler comprising the aerosol composition comprising aggregates of nanoparticulate drug particles, and/or nanoparticulate excipient particles, and optionally a binder, in admixture with one or more physiologically acceptable diluents or carriers.

The carrier or diluent particles, are of suitable particle size and size distribution, and may include such materials as lactose, mannitol or starch. In suitable embodiments, the pharmaceutical formulation may further include a lubricant, chemical stabilizer or physical stabilizer, such as magnesium stearate, sodium stearate or calcium stearate.

When diluent/carrier particles, such as lactose, are employed, generally, the particle size of the diluent/carrier excipient will be larger than 10 microns. For example, lactose particles may have a mass median diameter of 50-90 μm.

The present invention is also directed to aggregate particles for use in dry powder and/or propellant based aerosol drug delivery systems, wherein the aggregate particles comprise
(a) nanoparticulate drug particles and/or
(b) nanoparticulate excipient particles, and,
(c) optionally, binder,
wherein the nanoparticulate drug and/or excipient particles have a pre-selected crystalline form.

In certain embodiments, particularly where the nanoparticle content of the aggregate is is only drug, the aggregate preferably substantially free of a suspension homogenizing surfactant.

In one embodiment, the present invention is directed to dry powder and propellant based aerosol formulations containing aggregate particles, wherein the aggregate particles include nanoparticulate drug particles and nanoparticulate excipient particles, and, optionally, binder.

Preferably, in one embodiment, the aggregate particles are about 100 microns in aerodynamic diameter or less, such as 50 microns or less, whereas the nanoparticulate drug and nanoparticulate excipient particles are less than 1000 nm.

The aggregates of the nanoparticulate drug and nanoparticulate excipient particles may be designed for deposition to a desired location in the pulmonary system. Thus, the dry powder aerosol composition made up of the aggregates has an average mass mean aerodynamic diameter of 100 microns (μm) or less. The aggregate particles may be created having a specific particle size range to allow for the desired deposition behavior.

Aggregates to be deployed for alveolar region delivery have a mass median aerodynamic diameter of less than about 3 microns. For example, the compositions for alveolar delivery have a mass median aerodynamic diameter from 1 to 3 microns, for example from about 1 to 2 microns.

Aggregates for topical delivery to the bronchiole region of lung may be formed to a mass median aerodynamic diameter of less than 10 microns, for example, from about 3 to about 10 microns, such as from about 3 to about 6 microns, for example, from approximately 4 to about 5 microns.

Particle compositions for deposition in the upper regions of the pulmonary system may be produced to have a mass median aerodynamic diameter of greater than 10 microns, such as from 10 to about 100 microns.

Suitable aggregates may be generally spherical or irregular. The aggregate particle surface is suitably rough, to afford reduced particle-to-particle adhesion. The aggregates are held together by Van der Waals forces between adjacent nanoparticles, mechanical interlocking of nanoparticles, capillary adhesion and/or bridging between nanoparticles due to precipitation of dissolved materials.

In certain preferred embodiments of the invention, the nanoparticulate drug particles are substantially crystalline (as tested from sampling from the non-aqueous dispersion prior to aggregate particle formation), and/or in the aggregate particles themselves. Alternatively, the nanoparticulate excipient particles are substantially crystalline in non-aqueous dispersion and/or in the aggregate. In still further embodiments, both the nanoparticulate drug particles and the nanoparticulate excipient particles are substantially crystalline in dispersion and in the aggregate.

In instances where nanoparticulate drug particles comprise nanoparticles of different drugs, i.e., more than one type of active pharmaceutical ingredients, some or all of the different drugs may be substantially crystalline. Most preferably, each drug in the aggregate particles is substantially crystalline. Similarly, in instances where the nanoparticulate excipient particles comprise nanoparticles of different excipients, some or all of the different excipients may be substantially crystalline. Most preferably, all excipient nanoparticles are substantially crystalline prior to spray drying of aggregate particles.

It is preferred in some embodiment, that the crystalline form of the nanoparticle drug and/or excipient are each preselected, and that the preselected crystalline form of the drug/excipient is the same before and after aggregate particle formation.

The aerosol composition according to the present invention includes one or more drugs, in the form of drug nanoparticles. Suitable drug substances can be selected from a variety of known therapeutic classes of drugs, including but not limited to, ace-inhibitors, alpha-adrenergic agonist, beta-andrenergic agonists, alpha-andrenergic blockers, beta-andrenergic blockers, andrenocortocoidal steroids, andrenocortical supressors, adrenocorticotropic hormones, alcohol deterrents, aldose reductase inhibitors, aldosterones antagonists, 5-alpha reductase inhibitors, AMPA receptor antagonists, anobolocs, analeptics, analgesics (dental, narcotic and non-narcotic), androgens, anesthetics (inhalation, intravenous, local), angiotension converting ezyme inhibitors, angiotension II receptor antagonists, anorexics, antacids, anthelmintics, antiacanes, antiallegics (inculuding, antihistaminics, decongestants, glucocoiticoids), antialopecia agents, antiaembics, antiandrogens antianginals, antiarrhythmics, antiarteriosclerotics, antiarthritics/antirheutics, antiastmatic, antibactertials, anticancer agents, anticholinergics, anticonvulsants, antidepressants, antidiabetics, antidiarrheals, antidiuretics, antidyskenetics (including antiparkinsonian agentss), anti-emetics, antiestrogens, antifungals, antiglaucoma agents, antihistaminics, antihypertensives, antiinflammatories (both steroidal and non-steroidal), antimalarials, antimigraine, anti-muscarinics, antineutropenics, antiobesity agents, antiobsessionals, antiprotozoals, antipsychotics, antipyretics, antispasmodics, antivirals, bronchodilators, cholinegics, cns stimulants, contraceptives, vasodilators (including coronary vasodilators), decongestants, diagnostic aids, diuretics, dopamines receptor agonists, elastase inhibitors, expectorants, glucoicorticoids, histamine receptor antagonist, HIV inhibitors, leukotriene antagonists, sedatives/hypnotics, vasodilators, alone or in any combination.

A description of these classes of drugs and a listing of species within each class can be found in Martindale, the Extra Pharmacopeia, The Pharmaceutical Press, London.

Particularly preferred classes of drugs include, analgesics, anti-cholinergic agents, anti-inflammatory agents, antihistamines, anti-muscarinic agents, beta-adrenoceptor blocking agents, bronchodilators, corticosteroids, cough suppressants, (expectorants and mycoylitics), p38 kinase inhibitors, PDE4 modulators, IKK2 modulators, alone or in any combination.

Combination therapies are also considered within the scope of the invention, for example, aggregates may formed containing one or more corticosteroids, bronchodilator, anticholinergic agents, p38 kinase inhibitors, PDE4 modulators, IKK2 moduatiors and anti-muscarinic agents, or any combination thereof.

Particularly suitable combinations include combinations of beta agonists and corticosteroids, such as salmeterol and fluticasone propionate, salmeterol xinafoate and fluticasone propionate, vilanterol trifenatate and fluticasone furoate, mometasone furoate and formoterol fumarate, formoterol fumarate (and solvates thereof, including the dehydrate) and budesonide; formoterol and fluticasone propionate.

Suitable drugs include but are not limited to beclomethasone dipropionate, fluticasone propionate, salmeterol, salmeterol hydroxynapthanoate, fluticasone furoate, vilanterol, vilanterol trifenatate. In certain preferred embodiments, the drug is beclomethasone dipropionate, fluticasone propionate, salmeterol, salmeterol hydroxynapthanoate, fluticasone furoate, vilanterol, vilanterol trifenatate, alone or any combination thereof.

The invention also relates to a method of administering an aerosol composition as described herein, to a patient, wherein the aerosol comprises drug at a concentration of 0.1 mg/g or greater.

The aerosol composition suitably has a concentration of a drug in an amount of from about 0.005 mg/g powder up to about 1000 mg/g powder. For example, the aerosol composition may possess a concentration of a drug such as about 0.05 mg/g or more, 0.5 mg/g or more, 1 mg/g or more, 5 mg/g or more, 10 mg/g or more, 25 mg/g or more, 50 mg/g or more, or about 100 mg/g or more, about 200 mg/g or more, about 400 mg/g or more, about 600 mg/g or more, 800 mg/g or more, and about 1000 mg/g. Concentration of drug in the powder is drug potency dependant, and may be selected accordingly.

One or more excipient materials may be used to make up the excipient nanoparticles, making up the non-aqueous dispersion and resulting aggregate particles. Suitably excipients useful in the invention include, but are not limited to, amino acids, sugars, poly(amino acids), stearates, sugars, fatty acid esters, sugar alcohols, cholesterol, cyclodextrins and innon-aqueous molecules, and any combination thereof.

Suitable amino acids include, for example, leucine, iso-leucine, valine, and glycine, or any combination thereof. Suitable sugars include, for example, lactose, sucrose, glucose and trehalose or any combination thereof. Preferred polyamino acids include trileucine. Suitable stearates include, for example, magnesium stearate, sodium stearate and/or calcium stearate. Suitable sugar alcohols include, for example, mannitol, sorbitol, inositol, xylitol, erythritol, lactitol, and malitol, or any combination thereof. Suitable excipients also include cyclodextrins, EDTA, ascorbic acid, Vitamin E derivatives, di-keto-piperazine, taste masking agents, aspartame, sucralose, and citric acid and its salts, or any combination thereof. Suitable inorganic materials include, for example, sodium chloride, calcium chloride, one or more carbonate, or one or more phosphate, or any combination thereof. Suitable inorganic materials include, for example, carbonates, such as potassium carbonate, calcium carbonate, magnesium carbonate, and ammonium carbonate, or any combination thereof. Suitable inorganic materials may also include phosphates, for example, sodium phosphate, potassium phosphate and calcium phosphate, alone or in combination.

When employed, the optional binder in aggregates may include one or more polymers, dextrans, substituted dextrans, lipids, and/or surfactants. Polymeric binders include, but are not limited to PLGA, PLA, PEG, chitosan, PVP, PVA, hyaluronic acid, DPPC, and DSPC or any combination thereof. In certain instances, the binder is selected from the group consisting PLGA, PLA, PEG, chitosan, PVP, PVA, hyaluronic acid, DPPC, and DSPC or any combination thereof. In certain preferred embodiments, the binder is selected from the group consisting lecithin, DPPC and/or DSPC.

The binder may also comprise a quantity of the excipient of the excipient nanoparticles which dissolves in the non-aqueous liquid prior to aggregate formation.

The non-aqueous liquid in which the drug and excipient particles are dispersed prior to drying (and/or during nanoparticle creation) can be any non-aqueous media desired, having appropriate characteristics for its intended use, as would be readily determinable by those of ordinary skill.

Suitable non-aqueous dispersing media include, but are not limited to alcohols, ketones, esters, alkanes (linear or cyclic), chlorinated hydrocarbons, fluorinated hydrocarbons, ethers, either alone or mixtures of thereof. Particularly suitable non-aqueous liquid media include the alcohols, ethanol and propanol. Particularly suitable ketones include acetone and methylethylketone. Suitable esters include ethyl acetate and isopropylacetate. Suitable alkanes include isooctane, cyclohexane and methylcyclohexane. Suitable chlorinated hydrocarbons include p11 and p12. Suitable fluorinated hydrocarbons include p134a and p227. Suitable ethers include methyl-tert-butyl ether (MTBE), cyclopentyl-methyl-ether (CPME), Mixtures of various dispersing media are considered to be within the scope of the invention, including mixtures of the classes of media listed above, to achieve poor solubility of the drug and excipient.

The invention also relates to a formulation of a dry powder aerosol composition for use in a propellant-based pMDI comprising a powder composition as described herein formulated with a non-aqueous propellant. Suitably, the propellant is a non-CFC propellant. The invention also relates to a dry powder aerosol composition for use in a DPI.

### DETAILED DESCRIPTION OF THE INVENTION

### Definitions

The following terms have the meanings indicated below, unless otherwise specifically indicated or an alternative meaning is clear from the manner or context in which the terms is used.

As used herein “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

“Aggregate particle” means a composite particle which include one or more nanoparticles. The terms “aggregate particle” and “aggregate” are used interchangeably herein, unless an alternative meaning is clearly identified or is apparent from the context in which the given term is used.

“Binder” mean a material which assists in the maintaining the structural integrity of the individual aggregate particles.

Throughout the specification and the claims which follow, unless the context requires otherwise, the word ‘comprise’, and variations such as ‘comprises’ and ‘comprising’, will be understood to imply the inclusion of a stated integer or step or group of integers but not to the exclusion of any other integer or step or group of integers or steps.”

Throughout the specification and the claims which follow, unless the context requires otherwise, the word ‘consist’, and variations such as ‘consists’ and ‘consisting’, will be understood to imply the stated integer or step or group of integers to the exclusion of any other integer or step or group of integers or steps.

Throughout the specification and the claims which follow, unless the context requires otherwise, the word ‘consist essentially of’, and variations such as ‘consists essentially of’ and ‘consisting essentially of’, will be understood to imply the stated integer or step or group of integers to the exclusion of any essential significant other integer or step or group of integers or steps to the claimed subject matter.” The term “consisting essentially” of is to be understood as narrower than the transitional term “comprising” but broader than the term “consisting.”

“Drug” shall mean a material having a therapeutic or prophylactic effect in the treatment or prophylaxis of a disease or condition. The terms “drug”, “medicament”, “active pharmaceutical agent (“API”)” and “active agent” or are used interchangeably herein.

“Dry Powder Inhaler (DPI)” means a delivery device which contains a one or more doses of a dry powdered drug formulation, and which is capable of delivering a dose of the dry powdered drug to a patient.

“Excipient” shall mean a material which is incorporated in a composition for reasons other than the therapeutic or prophylactic effect of the excipient material in question.

“Homogenizing surfactant” means a compound which is dissolved in the non-aqueous liquid dispersing media that reduces the interfacial tension between the liquid and the solid materials dispersed in the liquid media and is used during size reduction processes, e.g. bead milling.

“Mass Median Aerodynamic Diameter”: The median of the distribution of airborne particle mass with respect to the aerodynamic diameter, e.g., as measured e.g. by cascade impaction.

“Mass Median Diameter”: The median size of a population of particles by mass, where 50% of the particles are above this diameter and 50% are below this diameter, e.g., as determined by laser diffraction, e.g., Malvern, Sympatec

“Metered Dose Inhaler (MDI)” means a drug delivery device which includes a canister, a formulation within the canister comprising a propellant formulation including, but not limited to, a drug suspended in a liquid propellant, where the canister is fitted with a metering valve for metering a quantity of the formulation, and actuator for releasing the metered quantity, and a mouthpiece or nose piece through which a patient inhales the dose released by the actuator.

“Nanoparticulate” shall mean a particle having a size less than 1 micron, unless otherwise specified or clear from the context in which the context in which it is used. Nanoparticulate and nanoparticle are used interchangeably herein. For example, the nanoparticles suitably have an effective average particle size less than about 800 nm, such as less than 600 nanometers, such as suitably less than 400 nm, or suitably less than about 300 nm, or suitably less than about 250 nm, or suitably less than about 100 nm, or suitably less than about 50 nm.

“Non-aqueous liquid”: means a substance which is a liquid other than water (e.g., an organic liquid). An “organic liquid” as used herein is a material which is in a liquid phase at a selected temperature and pressure and which contains at least one carbon atom.

“Non-solvent”: means a liquid in which a given solid material is insoluble, or is only marginally soluble (e.g., less than 10 mg/ml, e.g. 8 mg/ml or less, 7 mg/ml or less, 6 mg/ml or less, 5 mg/ml or less, 4 mg/ml or less, 3 mg/ml or less, 2 mg/ml or less, 1 mg/ml or less, 0.5 mg/ml or less, 0.1 mg/ml or less, 0.01 mg/ml or less, or 0.005 mg/ml or less) such that the liquid is capable of having the solid material be suspended therein in nanoparticulate form.

“Particle Size Distribution” (PSD) means the distribution of the size of particles as determined by suitable analysis, such as wet laser diffraction, (e.g., Malvern, Sympatec, etc.).

“Pre-selected crystalline form” means the desired crystalline form possessed by a sample of material prior to aggregate particle formation, as determined e.g., by XRPD.

Powder aerosol composition: means a quantity of a powder which includes aggregate particles.

As used herein, words expressed in the singular, shall be understood to mean the plural, unless otherwise clearly indicated to the contrary. Thus, the term “drug” means one or more drugs, unless otherwise specified or it is clear from the context in which the term is used. The term “excipient” means one or more excipients, unless otherwise specified or it is clear from the context in which the term is used, and the term “binder” means one or more binders, unless otherwise specified or it is clear from the context in which the term is used.

Further to the prior description, the present invention is also directed to aggregate particles for use in dry powder and/or propellant based aerosol drug delivery systems, wherein the aggregate particles comprise
(a) nanoparticulate drug particles and/or
(b) nanoparticulate excipient particles, and,
(c) optionally, binder,
wherein the nanoparticulate drug and/or excipient particles have a pre-selected crystalline form, and wherein the aggregate is substantially free of a suspension homogenizing surfactant.

In one embodiment, the invention is directed to dry powder aerosols of aggregate particles made up of nanoparticulate drug particles and nanoparticulate excipients particles, and, optionally, a binder.

The dry powder aerosol formulations comprising aggregate particles are for inhalation drug delivery, being adaptable to pulmonary and nasal administration. Thus, dry powders, which can be used in both DPIs and pMDIs, can be made by spray drying nanoparticulate drug and nanoparticulate excipient dispersed in a non-aqueous dispersing media. In this invention, “dry” refers to a composition having less than about 5% non-aqueous residue.

In a preferred embodiment, the aggregates are formed by spray drying from a non-aqueous dispersion of the nanoparticulate drug particles and nanoparticulate excipient particles in a non-aqueous liquid. The nanoparticulate drug particles and nanoparticulate excipient particles are suitably “poorly soluble” in the non-aqueous dispersing media, having solubility in the non-aqueous liquid of less than about 10 mg/ml.

Preferably, the aggregate particles are about 100 microns in aerodynamic diameter or less, whereas the nanoparticulate drug and nanoparticulate excipient particles are less than 1000 nm.

The Mass Median Aerodynamic Diameter (MMAD) of the composition of aggregates will depend on the intended deposition site of the aggregate particles. For example, for delivery of aggregate particles to the alveolar region of the lung, aerodynamically smaller aggregates may be employed. In such cases, the aggregates of the nanoparticulate drug and nanoparticulate excipient particles will be designed to have a mass median aerodynamic diameter of less than about 3 microns, although the exact size will depend on the selected air flow rate for deposition.

Composition of aggregates for topical delivery to the smooth muscle region of the lung, desirably have a Mass Median Aerodynamic Diameter of from about 1 to about 10 microns, such as from about 3 to about 6 microns, e.g., as from 4 to 5 microns.

Particles intended for deposition in the throat or nasal mucosa desirably have an aerodymanic diameter larger than 10 microns.

As aerodynamic properties of a particle are dependent in part by its relative density, the parameters of the spray drying process may be may be adjusted, along with the feed stock makeup, to produce particles having desired morphologies and relative densities. For example, dense, relatively solid, particles, i.e., particles having very little porosity or containing a low volume of internal opening(s) or cavities, may be produced having high relative densities.

In certain embodiments the particles may be non-solid. For example they may result in particles in which the nanoparticles have formed interconnections to create an outer shell surrounding an inner void(s), thus forming hollow particles.

In still other embodiments, the respirable particles may be porous throughout, defining a great number of interconnected passageways. As a general matter, the density and surface area affect the aerosolization behavior of a particle.

Another feature effecting both particle density and surface area is the external morphology of the particles. Thus particles generated using the methods described above may have a smooth, a rough or a porous or fissured external surface.

In certain embodiments, the aggregate particles produced may have densities which are less than 1 g/cm³, such that their aerodynamic diameter (as measured by the Mass Median Aerodynamic Diameter (MMAD, measured by cascade impaction) of the particle composition is less than their average geometric diameters, or Mass Median Diameter (MMD, measured by laser diffraction, such as a Malvern laser diffraction apparatus). For example, particle compositions having MMADs 1 and 10 microns, may well have MMD (geometric diameters) well over 10 microns, where the aggregates may be hollow or porous. A rough surrogate for particle density is tap density, i.e., the measured density of a quantity of powder containing within a graduated cylinder after compaction by tapping a set number of times. The tapped density of certain particle compositions considered to be “low density” is less than 0.5 g/cm³, such as 0.4 g/cm³, for example 0.2 g/cm³, such as 0.1 g/cm³.

In a preferred embodiment, it is desirable that the particles of respirable size have a low enough density to have enhanced aerosolization performance, but not a particle density so low that the particle cannot survive manufacturing processes, such as cyclone collection, or be filled using an dry powder inhaler filling platform, for example platforms where a blister is immersed in a bed of powder, or where powder is metered into a dosing cup, and then delivered to a blister to be filled.

As can be appreciated, the process described provides advantages relating to reducing manufacturing complexity (number of unit operations) in comparison with micronized drug powder blends, while still increasing the delivery efficiency of the drug substance to the lungs. One objective of this invention is to generate a formulation platform based on stable formulated particles that aerosolize efficiently. In one embodiment, such a formulation would not require the aggregate particles to be blended with a coarse carrier, such as non-respirable milled lactose, with or without further excipients.

Alternatively, in preparing pharmaceutical formulations for delivery with a DPI, admixing such agglomerate particles with further carrier or diluent particles (and other excipient materials) may further improve the aerosol performance in terms of the fraction of the drug delivered from a DPI, or the fraction of the drug delivered to the desired region of the pulmonary system. These dry powder formulations are considered within the scope of the present invention.

While aggregate particles as described herein may be of any morphology, in certain embodiments, the aggregate particles are generally spherical. Moreover, the particle surface may be smooth or roughened, porous or non-porous. In certain embodiments, the surface of the aggregate particles is preferably rough, to minimize contact surface between adjacent particles, and also possibly adding to the improved aerodynamic behavior of such particles when entrained in an airflow stream.

The Applicants believe that because the aggregate particle composition relies on nanoparticles, the invention process lends itself to simplified dose ranging studies. In this system, the percentage of nanoparticulate drug may be increased relative to the percentage of nanoparticulate excipient particles and the overall morphology of the resulting particles should not be significantly affected. Moreover, the aerodynamic qualities should remain relatively constant between particle compositions of different potencies.

Furthermore, nanoparticles may be fabricated and maintained in crystalline form for both drug(s) and excipient(s) through the spray drying process. Crystallinity may be determined by Powder X-Ray diffraction. Thus, greater stability is afforded in the use of spray dried respirable sized particles containing crystalline nanoparticles, than in other drug product presentations, such as micronized blend formulations or formulations containing amorphous particles.

The aggregate particles of the present invention comprise nanoparticulate drug particles and nanoparticulate excipient particles, and, optionally, binder (It is to be understood that “drug” as used herein includes one drug, or more than one drug; “excipient” included one excipient, or more than one excipient; and “binder” means one binder or more than one binder).

As will be appreciated, the relative amount of drug, excipient and optional binder can vary widely, depending on the materials selected. The optimal amount of the excipient to drug, or binder to excipient and drug can depend upon, for example, on the particular drug(s), the particular excipient(s) selected, the propensity of the drug(s) and excipients(s) to form suitable aggregates up on spray drying, the solubility of the drug(s) or excipient(s) in the non-aqueous media, etc.

The aggregate particles generally contain less than 100% drug w/w aggregate, 0 to 99.99% excipient w/w aggregate. For example, the drug may represent 99.999% of the aggregate particles where the non-drug (excipient) fraction is 0.001% of the aggregate particles; the drug may represent 99% of the aggregate particles where the non-drug fraction is 1% of the aggregate particles; the drug may represent 95% of the aggregate particles where the non-drug fraction is 5% of the aggregate particles; the drug may represent 90% of the aggregate particles where the non-drug fraction is 10% of the aggregate particles; the drug may represent 85% of the aggregate particles where the non-drug fraction is 15% of the aggregate particles; the drug may represent 80% of the aggregate particles where the non-drug fraction is 20% of the aggregate particles; the drug may represent 75% of the aggregate particles where the non-drug fraction is 25% of the aggregate particles; the drug may represent 70% of the aggregate particles where the non-drug fraction is 30% of the aggregate particles; the drug may represent 65% of the aggregate particles where the non-drug fraction is 35% of the aggregate particles; the drug may represent 60% of the aggregate particles where the non-drug fraction is 40% of the aggregate particles; the drug may represent 55% of the aggregate particles where the non-drug fraction is 45% of the aggregate particles; the drug may represent 50% of the aggregate particles where the non-drug fraction is 50% of the aggregate particles; the drug may represent 45% of the aggregate particles where the non-drug fraction is 55% of the aggregate particles; the drug may represent 40% of the aggregate particles where the non-drug fraction is 60% of the aggregate particles; the drug may represent 35% of the aggregate particles where the non-drug fraction is 65% of the aggregate particles; the drug may represent 30% of the aggregate particles where the non-drug fraction is 70% of the aggregate particles; the drug may represent 25% of the aggregate particles where the non-drug fraction is 75% of the aggregate particles; the drug may represent 20% of the aggregate particles where the non-drug fraction is 80% of the aggregate particles; the drug may represent 15% of the aggregate particles where the non-drug fraction is 85% of the aggregate particles; the drug may represent 10% of the aggregate particles where the non-drug fraction is 90% of the aggregate particles; the drug may represent 5% of the aggregate particles where the non-drug fraction is 95% of the aggregate particles; the drug may represent 1% of the aggregate particles where the non-drug fraction is 99% of the aggregate particles; the drug may represent 0.5% of the aggregate particles where the non-drug fraction is 99.5% of the aggregate particles; the drug may represent 0.05% of the aggregate particles where the non-drug fraction is 99.95% of the aggregate particles; the drug may represent 0.001% of the aggregate particles where the non-drug fraction is 99.999% of the aggregate particles.

The non-drug fraction of the aggregate particles may be 100% excipient and 0% binder. Where a binder is present though, the binder may comprise from 99.99% to 0.001% of the non-drug fraction of the aggregate particles. In certain embodiments, the binder is 50% or less w/w aggregate, for example 40% or less, 30% or less, 20% or less, 10% or less, 5% or less, 1% or less, 0.5% or less, 0.05% or less, or 0.001% or less w/w of the aggregate particles.

In one aspect of an embodiment of the invention, the binder is a small amount of the drug or the excipient making up the nanoparticles that has been dissolved in the suspending media. In these embodiments, the drug or excipient is dissolved either in the liquid non-solvent (hence the liquid non-solvent is actually a sparse solute for the suspended drug or excipient).

Alternatively, a small amount of active or excipient may be dissolved in a separate liquid or co-solvent system, and intimately mixed with the suspending liquid either prior to or simultaneously upon formation of droplets which when dried form the respirable particles containing the nanoparticles.

The binder can be included in the suspension liquid, can be added to the suspension immediately before spray drying, or can be supplied at the point of droplet generation, for example, via a co-axial nozzle. An advantage of the excipient binder is that it provides a generic surface to the particle allowing physical performance and chemical stability to be more predictable.

As used herein, “nanoparticles” have an effective average size of less than 1000 nm, preferably, as previously described, such as less than 800 nm, such as less than 600 nanometers, for example 400 nanometers or less, in some instances about 200 nanometers or less, etc.

Nanoparticles may be prepared in any conventional way. However, in one aspect of the present invention, nanoparticles are prepared in a bead milling device, such as the Cosmo DRIAS 2 bead mill. In the bead mill process, the material to be milled is placed in a liquid suspending media, preferably in a non-aqueous liquid. As previously mentioned, the materials to be milled should be generally insoluble in the non-aqueous liquid media. Preferred liquid media include ethyl acetate, isopropyl acetate, isooctane, cyclohexane or ethanol.

The bead mill is prepared with beads of a given material and bead size in a container of a suitable size. In a preferred embodiment, the beads used in the mill are nylon or yttrium stabilized zirconium oxide beads. Any suitable bead size may be employed in the milling chamber, for example 0.3 mm, or 0.4 mm beads. The suspension is re-circulated through milling chamber using a peristaltic pump. A suitably sized sieve screen may be employed in the bead mill, such as a 0.15 mm size sieve screen. Mill speed is selected to operate to the appropriate result, for example, at 80% of maximum. The suspension is thus milled and re-circulated until the particle size of the drug has been reduced to the desired size. Obviously, operating conditions for the bead mill may be selected in order to achieve the appropriate sized nanoparticles.

Excipient materials are those suitable to act as bulking agents or diluents in the composite particles or respirable size, as described elsewhere in this specification. Particularly suitable excipients include sugars such as lactose, sugar alcohols such as mannitol, inositol and erythritol, or amino acids, such as leucine, L-leucine and iso-leucine.

While drug and excipient nanoparticles may be milled separately in the non-aqueous bead mill process, in one embodiment of the present invention, both drug and excipient materials are bead milled in a common suspension. This “co-milling” approach advantageously provides intimate mixing of the nanoparticulate drug and the nanoparticulate excipient.

In certain embodiments, the milling process is conducted in the absence of a homogenizing surfactant in the liquid non-aqueous media. It has surprisingly been found that careful selection of liquid non-solvent can eliminate the need for the use of surfactant in the suspension undergoing bead milling. This provides a significant advantage in eliminating unnecessary additives to the process that may have to be removed later in the manufacturing process, and avoids the potential that residues of the used surfactant would be retained even after washing. Such surfactant may pose a possible toxicological issue, thus requiring it to be removed, for example by washing. Extracting the surfactant may be difficult, as residual surfactant may remain even after this washing/extraction process.

Thus, a further aspect of the of the present invention involves a method of forming a dispersion of nanoparticulate drug particles and nanoparticulate excipient particles in a non-aqueous liquid, by bead milling larger particles of drug and/or excipient in the non-aqueous dispersing non-aqueous liquid. Suitably, the drug and excipient which are milled to produce nanoparticulate particles are bead milled together in the liquid dispersing media.

Thus, the compositions of the invention contain nanoparticles of drug and of excipient which, independently, have an effective average particle size of less than about 1000 nm, more preferably less than about 400 nm, less than about 300 nm, less than about 250 nm, less than about 100 nm, or less than about 50 nm, as measured by light-scattering methods. By “an effective average particle size of less than about 1000 nm” it is meant that at least 50% of the drug particles have a weight average particle size of less than about 1000 nm when measured by light scattering techniques. Preferably, at least 70% of the drug particles have an average particle size of less than about 1000 nm, more preferably at least 90% of the drug particles have an average particle size of less than about 1000 nm, and even more preferably at least about 95% of the particles have a weight average particle size of less than about 1000 nm.

In certain suitable embodiments, at least 50% of the drug particles have a weight average particle size of less than about 400 nm when measured by light scattering techniques. Preferably, at least 70% of the drug particles have an average particle size of less than about 400 nm, more preferably at least 90% of the drug particles have an average particle size of less than about 400 nm, and even more preferably at least about 95% of the particles have a weight average particle size of less than about 400 nm.

The aggregate particles are formed from nanoparticulate drug particles and nanoparticulate excipient particles. Suitably, 50% or more of the nanoparticulate drug particles and 50% or more of the nanoparticulate excipient particles which make up the aggregate particles have an average particle size of less than 1000 nm.

The nanoparticulate measurements are determined as measured from the size reduced input materials in the non-aqueous liquid dispersion prior to aggregate formation. Because the nanoparticles of drug and excipient are poorly soluble in the non-aqueous media, the size of the nanoparticles is maintained through the aggregate formation process.

The nanoparticulate drug and nanoparticulate excipient particles suitably have an effective average particle size less than 1000 nm, for example, they are suitably less than about 400 nm, or suitably less than about 300 nm, or suitably less than about 250 nm, or suitably less than about 100 nm, or such as less than about 50 nm.

In one preferred embodiment, the nanoparticulate drug and/or excipient particles have an effective average particle size of less than about 300 nm. In another preferred embodiment, the nanoparticulate drug and/or excipient particles have an effective average particle size of less than about 250 nm. In still further embodiments, the nanoparticulate drug and/or excipient particles have an effective average particle size of less than about 100 nm. In still further alternative embodiments, the nanoparticulate drug and/or excipient particles have an effective average particle size of less than about 50 nm.

Suitably, in certain embodiments, at least 70% of the drug and excipient particles have a particle size of less than about 1000 nm, for example, suitably, at least 90% of the drug and excipient particles have a particle size of less than about 1000 nm. For example, at least 70% of the drug and excipient particles have a particle size of less than about 400 nm, for example, suitably, at least 90% of the drug and excipient particles have a particle size of less than about 400 nm.

In one embodiment of the invention, the nanoparticulate drug particles are substantially crystalline in the dispersion and in the aggregate particles.

In a further embodiment of the invention, the nanoparticulate excipient particles are substantially crystalline in the dispersion and in the aggregate.

In still further embodiments, both the nanoparticulate drug particles and the nanoparticulate excipient particles are substantially crystalline in dispersion and in the aggregate.

The aggregate particles may comprise nanoparticles of one of more different therapeutically active drugs. In instances where nanoparticulate drug particles comprise nanoparticles of different drugs, i.e., more than one type of active pharmaceutical ingredients, some or all of the different drugs may be substantially crystalline. Most preferably, each drug in the aggregate particles is substantially crystalline.

Similarly, in instances where the nanoparticulate excipient particles comprise nanoparticles of different excipients, some or all of the different excipients may be substantially crystalline. Most preferably, all excipient nanoparticles are substantially crystalline prior to spray drying of aggregate particles.

It is a potential advantage of the present invention that crystallinity and, therefore, physical stability of the aggregate composition may be maximized by reducing the amount of amorphous material in the aggregate particles by controlling and/or maintaining the crystallinity of the nanoparticulate starting materials. As is described herein, the nanoparticles are crystalline material and this crystallinity is maintained during the spray drying process. In the event that amorphous drug or excipient is formed during the spray drying process, the crystalline nanoparticles drug or excipient may act as seed materials to generate the desired crystalline form during aggregate formation. In addition, selected temperatures may be used during spray-drying which induces the amorphous-to-crystalline conversion. Further still, the spray dried material may be exposed to a gaseous solvent during or following aggregate formation to drive amorphous to crystalline conversion. Greater physical and/or chemical stability may be achieved in the resulting aggregate particle composition in this manner. Thus, the present invention affords the benefit of controlling the attributes of the crystal form of the aggregate particles and better quality control.

The compositions and pharmaceutical formulations according to the invention may include one or more other therapeutic agents.

The one or more therapeutic agents may be incorporated within an individual aggregate particle as nanoparticles. Alternatively, one type of aggregate particle may contain a single type of therapeutic agent, and be combined in a powder blend with another type of aggregate particle containing one or more different therapeutic agent(s). These different types of aggregate particles can be blended together and contained within a single container (e.g., a capsule or blister) for co-delivery within a single breath, or may be packaged in different containers within the same device, where the contents of containers may be accessed at same time for co-delivery within a single breath.

Suitable therapeutic agents for the compositions and formulations of the present invention, include but are not limited to, anti-inflammatory agents, anticholinergic agents (particularly an M₁, M₂, M₁/M₂ or M₃ receptor antagonist), β₂-adrenoreceptor agonists, antiinfective agents (e.g. antibiotics, antivirals), antihistamines, p38 kinase, PDE4, IKK2, and/or TRPV1 antagonist.

The invention thus may incorporate one or more anti-inflammatory agents (for example corticosteroids, Non-Steroidal Anti-Inflammatory Drugs (NSAIDs), anticholinergics; β₂-adrenoreceptor agonists, antiinfective agents (e.g. an antibiotic or an antiviral), antihistamines, p38 kinase inhibitors, PDE4, IKK2 modulators, and/or TRPV1 antagonist, either alone or in any combination.

Preferred combinations are those comprising two or three different therapeutic agents.

It will be clear to a person skilled in the art that, where appropriate, the other therapeutic ingredient(s) may be used in the form of salts, (e.g. as alkali metal or amine salts or as acid addition salts), or prodrugs, or as esters (e.g. lower alkyl esters), or as solvates (e.g. hydrates) to optimise the activity and/or stability and/or physical characteristics (e.g. solubility) of the therapeutic ingredient. It will be clear also that where appropriate, the therapeutic ingredients may be used in optically pure form.

One suitable combination of the present invention comprises an anti-inflammatory agent together with a β₂-adrenoreceptor agonist. Examples of β₂-adrenoreceptor agonists include vilanterol, salmeterol (which may be a racemate or a single enantiomer, such as the R-enantiomer), salbutamol, formoterol, salmefamol, indacaterol, fenoterol or terbutaline and salts thereof, for example the trifenatate salt or vilanterol, the xinafoate salt of salmeterol, the sulphate salt or free base of salbutamol or the fumarate salt of formoterol. Long-acting β₂-adrenoreceptor agonists are preferred, especially those having a therapeutic effect over a 24 hour period, such as glycopyrronium, vilanterol, or formoterol.

Suitable long acting β₂-adrenoreceptor agonists include those described in WO02/66422A, WO02/270490, WO02/076933, WO03/024439, WO03/072539, WO 03/091204, WO04/016578, WO04/022547, WO04/037807, WO04/037773, WO04/037768, WO04/039762, WO04/039766, WO01/42193 and WO03/042160, whose disclosures are incorporated by reference herein. Preferred long-acting β₂-adrenoreceptor agonists are:
3-(4-{[6-({(2R)-2-hydroxy-2-[4-hydroxy-3-(hydroxymethyl)phenyl]ethyl}amino) hexyl]oxy}butyl)benzenesulfonamide;
3-(3-{[7-({(2R)-2-hydroxy-2-[4-hydroxy-3-hydroxymethyl)phenyl]ethyl}-amino)heptyl]oxy}propyl)benzenesulfonamide;
4-{(1R)-2-[(6-{2-[(2,6-dichlorobenzyl)oxy]ethoxy}hexyl)amino]-1-hydroxyethyl}-2-(hydroxymethyl)phenol;
4-{(1R)-2-[(6-{4-[3-(cyclopentylsulfonyl)phenyl]butoxy}hexyl)amino]-1-hydroxyethyl}-2-(hydroxymethyl)phenol;
N-[2-hydroxyl-5-[(1R)-1-hydroxy-2-[[2-4-[[(2R)-2-hydroxy-2-phenylethyl]amino]phenyl]ethyl]amino]ethyl]phenyl]foramide, and
N-2{2-[4-(3-phenyl-4-methoxyphenyl)aminophenyl]ethyl}-2-hydroxy-2-(8-hydroxy-2(1H)-quinolinon-5-yl)ethylamine.

Suitable anti-inflammatory agents include corticosteroids. Suitable corticosteroids which may be used in combination with the compounds of the invention are those oral and inhaled corticosteroids and their pro-drugs which have anti-inflammatory activity. Examples include Flunisolide, fluticasone propionate, fluticasone furoate, 6α,9α-difluoro-11β-hydroxy-16α-methyl-3-oxo-17α-propionyloxy-androsta-1,4-diene-17β-carbothioic acid S-(2-oxo-tetrahydro-furan-3S-yl) ester, 6α,9α-difluoro-11β-hydroxy-16α-methyl-17α-(1-methylcylopropylcarbonyl)oxy-3-oxo-androsta-1,4-diene-17β-carbothioic acid S-fluoromethyl ester, 6α,9α-difluoro-11β-hydroxy-16α-methyl-3-oxo-17α-(2,2,3,3-tetramethylcyclopropylcarbonyl)oxy-androsta-1,4-diene-17β-carboxylic acid cyanomethyl ester, beclomethasone esters (such as the 17-propionate ester or the 17,21-dipropionate ester), budesonide, flunisolide, mometasone esters (such as the furoate ester), triamcinolone acetonide, rofleponide, ciclesonide, (16α,17-[[(R)-cyclohexylmethylene]bis(oxy)]-11β,21-dihydroxy-pregna-1,4-diene-3,20-dione), butixocort propionate, RPR-106541, and ST-126. Preferred corticosteroids include fluticasone propionate, 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester and 6α,9α-difluoro-17α-[(2-furanylcarbonyl)oxy]-11β-hydroxy-16α-methyl-3-oxo-androsta-1,4-diene-17β-carbothioic acid S-fluoromethyl ester, more preferably 6α,9α-difluoro-17α-[(2-furanylcarbonyl)oxy]-11β-hydroxy-16α-methyl-3-oxo-androsta-1,4-diene-17β-carbothioic acid S-fluoromethyl ester.

Non-steroidal compounds having glucocorticoid agonism that may possess selectivity for transrepression over transactivation and that may be useful in combination therapy include those covered in the following patents: WO03/082827, WO01/10143, WO98/54159, WO04/005229, WO04/009016, WO04/009017, WO04/018429, WO03/104195, WO03/082787, WO03/082280, WO03/059899, WO03/101932, WO02/02565, WO01/16128, WO00/66590, WO03/086294, WO04/026248, WO03/061651, WO03/08277.

Suitable anti-inflammatory agents include non-steroidal anti-inflammatory drugs (NSAID's). Suitable NSAID's include sodium cromoglycate, nedocromil sodium, phosphodiesterase (PDE) inhibitors (for example, theophylline, PDE4 inhibitors or mixed PDE3/PDE4 inhibitors), leukotriene antagonists, inhibitors of leukotriene synthesis (for example, montelukast), iNOS inhibitors, tryptase and elastase inhibitors, beta-2 integrin antagonists and adenosine receptor agonists or antagonists (for example, adenosine 2a agonists), cytokine antagonists (for example, chemokine antagonists, such as a CCR3 antagonist) or inhibitors of cytokine synthesis, or 5-lipoxygenase inhibitors. Suitable other β₂-adrenoreceptor agonists include salmeterol (for example, as the xinafoate), salbutamol (for example, as the sulphate or the free base), formoterol (for example, as the fumarate), fenoterol or terbutaline and salts thereof. An iNOS (inducible nitric oxide synthase inhibitor) is preferably for oral administration. Suitable iNOS inhibitors include those disclosed in WO93/13055, WO98/30537, WO02/50021, WO95/34534 and WO99/62875. Suitable CCR3 inhibitors include those disclosed in WO02/26722.

Another embodiment of the invention is the use of a phosphodiesterase 4 (PDE4) inhibitor or a mixed PDE3/PDE4 inhibitor. The PDE4-specific inhibitor useful in this aspect of the invention may be any compound that is known to inhibit the PDE4 enzyme or which is discovered to act as a PDE4 inhibitor, and which are only PDE4 inhibitors, not compounds which inhibit other members of the PDE family as well as PDE4.

Suitable PDE compounds are cis 4-cyano-4-(3-cyclopentyloxy-4-methoxyphenyl)cyclohexan-1-carboxylic acid, 2-carbomethoxy-4-cyano-4-(3-cyclopropylmethoxy-4-difluoromethoxyphenyl)cyclohexan-1-one and cis-[4-cyano-4-(3-cyclopropylmethoxy-4-difluoromethoxyphenyl)cyclohexan-1-ol].

Other compounds of interest include: Compounds set out in U.S. Pat. No. 5,552,438 issued 3 Sep. 1996; this patent and the compounds it discloses are incorporated herein in full by reference. The compound of particular interest, which is disclosed in U.S. Pat. No. 5,552,438, is cis-4-cyano-4-[3-(cyclopentyloxy)-4-methoxyphenyl]cyclohexane-1-carboxylic acid (also known as cilomalast) and its salts, esters, pro-drugs or physical forms; AWD-12-281 from elbion (Hofgen, N. et al. 15th EFMC Int. Symp. Med. Chem. (September 6-10, Edinburgh) 1998, Abst. P.98; CAS reference No. 247584020-9); a 9-benzyladenine derivative nominated NCS-613 (INSERM); D-4418 from Chiroscience and Schering-Plough; a benzodiazepine PDE4 inhibitor identified as CI-1018 (PD-168787) and attributed to Pfizer; a benzodioxole derivative disclosed by Kyowa Hakko in WO99/16766; K-34 from Kyowa Hakko; V-11294A from Napp (Landells, L. J. et al. Eur Resp J [Annu Gong Eur Resp Soc (September 19-23, Geneva) 1998] 1998, 12 (Suppl. 28): Abst P2393); roflumilast (CAS reference No 162401-32-3) and a pthalazinone (WO99/47505, the disclosure of which is hereby incorporated by reference) from Byk-Gulden; Pumafentrine, (−)-p-[(4aR*,10bS*)-9-ethoxy-1,2,3,4,4a,10b-hexahydro-8-methoxy-2-methylbenzo[c][1,6]naphthyridin-6-yl]-N,N-diisopropylbenzamide which is a mixed PDE3/PDE4 inhibitor which has been prepared and published on by Byk-Gulden, now Altana; arofylline under development by Almirall-Prodesfarma; VM554/UM565 from Vernalis; or T-440 (Tanabe Seiyaku; Fuji, K. et al. J Pharmacol Exp Ther, 1998, 284(1): 162), and T2585. Other possible PDE-4 and mixed PDE3/PDE4 inhibitors include those listed in WO01/13953, the disclosure of which is hereby incorporated by reference.

Suitable anticholinergic agents are those compounds that act as antagonists at the muscarinic receptor, in particular those compounds which are antagonists of the M₁ and M₂ receptors. Exemplary compounds include the alkaloids of the belladonna plants as illustrated by the likes of atropine, scopolamine, homatropine, hyoscyamine; these compounds are normally administered as a salt, being tertiary amines. These drugs, particularly the salt forms, are readily available from a number of commercial sources or can be made or prepared from literature data via, to with:

Atropine—CAS-51-55-8 or CAS-51-48-1 (anhydrous form), atropine sulfate—CAS-5908-99-6; atropine oxide—CAS-4438-22-6 or its HCl salt—CAS-4574-60-1 and methylatropine nitrate—CAS-52-88-0; Homatropine—CAS-87-00-3, hydrobromide salt—CAS-51-56-9, methylbromide salt—CAS-80-49-9; Hyoscyamine (d,l)-CAS-101-31-5, hydrobromide salt—CAS-306-03-6 and sulfate salt—CAS-6835-16-1; and Scopolamine—CAS-51-34-3, hydrobromide salt—CAS-6533-68-2, methylbromide salt—CAS-155-41-9.

Suitable anticholinergics for use herein include, but are not limited to, ipratropium (e.g. as the bromide), sold under the name Atrovent, oxitropium (e.g. as the bromide) and tiotropium (e.g. as the bromide) (CAS-139404-48-1). Also of interest are: methantheline (CAS-53-46-3), propantheline bromide (CAS-50-34-9), anisotropine methyl bromide or Valpin 50 (CAS-80-50-2), clidinium bromide (Quarzan, CAS-3485-62-9), copyrrolate (Robinul), isopropamide iodide (CAS-71-81-8), mepenzolate bromide (U.S. Pat. No. 2,918,408), tridihexethyl chloride (Pathilone, CAS-4310-35-4), and hexocyclium methylsulfate (Tral, CAS-115-63-9). See also cyclopentolate hydrochloride (CAS-5870-29-1), tropicamide (CAS-1508-75-4), trihexyphenidyl hydrochloride (CAS-144-11-6), pirenzepine (CAS-29868-97-1), telenzepine (CAS-80880-90-9), AF-DX 116, or methoctramine, and the compounds disclosed in WO 01/04118, the disclosure of which is hereby incorporated by reference.

Other suitable anticholinergic agents include compounds of formula (XXI), which are disclosed in U.S. patent application 60/487,981:

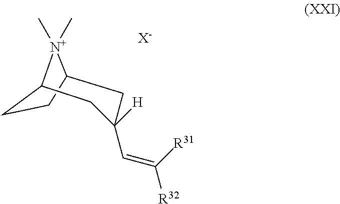

in which the preferred orientation of the alkyl chain attached to the tropane ring is endo;

R³¹ and R³² are, independently, selected from the group consisting of straight or branched chain lower alkyl groups having preferably from 1 to 6 carbon atoms, cycloalkyl groups having from 5 to 6 carbon atoms, cycloalkyl-alkyl having 6 to 10 carbon atoms, 2-thienyl, 2-pyridyl, phenyl, phenyl substituted with an alkyl group having not in excess of 4 carbon atoms and phenyl substituted with an alkoxy group having not in excess of 4 carbon atoms;
X⁻ represents an anion associated with the positive charge of the N atom.

X⁻ includes, but is not limited to chloride, bromide, iodide, sulfate, benzene sulfonate, and toluene sulfonate. Suitably, this includes the following exemplifications:
(3-endo)-3-(2,2-di-2-thienylethenyl)-8,8-dimethyl-8-azoniabicyclo[3.2.1]octane bromide;
(3-endo)-3-(2,2-diphenylethenyl)-8,8-dimethyl-8-azoniabicyclo[3.2.1]octane bromide;
(3-endo)-3-(2,2-diphenylethenyl)-8,8-dimethyl-8-azoniabicyclo[3.2.1]octane 4-methylbenzenesulfonate;
(3-endo)-8,8-dimethyl-3-[2-phenyl-2-(2-thienyl)ethenyl]-8-azoniabicyclo[3.2.1]octane bromide; and/or
(3-endo)-8,8-dimethyl-3-[2-phenyl-2-(2-pyridinyl)ethenyl]-8-azoniabicyclo[3.2.1]octane bromide.

Further suitable anticholinergic agents include compounds of formula (XXII) or (XXIII), which are disclosed in U.S. patent application 60/511,009:

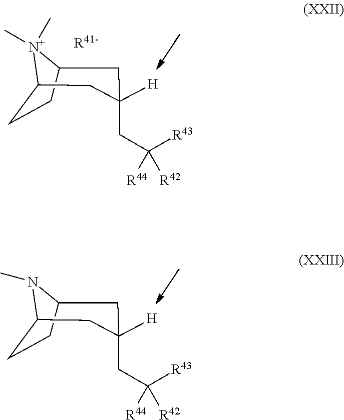

wherein:
the H atom indicated is in the exo position;
R⁴¹ represents an anion associated with the positive charge of the N atom, R⁴¹ may be, but is not limited to, chloride, bromide, iodide, sulfate, benzene sulfonate and toluene sulfonate;
R⁴² and R⁴³ are independently selected from the group consisting of straight or branched chain lower alkyl groups (having preferably from 1 to 6 carbon atoms), cycloalkyl groups (having from 5 to 6 carbon atoms), cycloalkyl-alkyl (having 6 to 10 carbon atoms), heterocycloalkyl (having 5 to 6 carbon atoms) and N or O as the heteroatom, heterocycloalkyl-alkyl (having 6 to 10 carbon atoms) and N or O as the heteroatom, aryl, optionally substituted aryl, heteroaryl, and optionally substituted heteroaryl;
R⁴⁴ is selected from the group consisting of (C₁-C₆)alkyl, (C₃-C₁₂)cycloalkyl, (C₃-C₇)heterocycloalkyl, (C₁-C₆)alkyl(C₃-C₁₂)cycloalkyl, (C₁-C₆)alkyl(C₃-C₇)heterocycloalkyl, aryl, heteroaryl, (C₁-C₆)alkyl-aryl, (C₁-C₆)alkyl-heteroaryl, —OR⁴⁵, —CH₂OR⁴⁵, —CH₂OH, —CN, —CF₃, —CH₂O(CO)R⁴⁶, —CO₂R⁴⁷, —CH₂NH₂, —CH₂N(R⁴⁷)SO₂R⁴⁵, —SO₂N(R⁴⁷)(R⁴⁸), —CON(R⁴⁷)(R⁴⁸), —CH₂N(R⁴⁸)CO(R⁴⁶), —CH₂N(R⁴⁸)SO₂(R⁴⁶), —CH₂N(R⁴⁸)CO₂(R⁴⁵), —CH₂N(R⁴⁸)CONH(R⁴⁷);
R⁴⁵ is selected from the group consisting of (C₁-C₆)alkyl, (C₁-C₆)alkyl(C₃-C₁₂)cycloalkyl, (C₁-C₆)alkyl(C₃-C₇)heterocycloalkyl, (C₁-C₆)alkyl-aryl, (C₁-C₆)alkyl-heteroaryl;
R⁴⁶ is selected from the group consisting of (C₁-C₆)alkyl, (C₃-C₁₂)cycloalkyl, (C₃-C₇)heterocycloalkyl, (C₁-C₆)alkyl(C₃-C₁₂)cycloalkyl, (C₁-C₆)alkyl(C₃-C₇)heterocycloalkyl, aryl, heteroaryl, (C₁-C₆)alkyl-aryl, (C₁-C₆)alkyl-heteroaryl;
R⁴⁷ and R⁴⁸ are, independently, selected from the group consisting of H, (C₁-C₆)alkyl, (C₃-C₁₂)cycloalkyl, (C₃-C₇)heterocycloalkyl, (C₁-C₆)alkyl(C₃-C₁₂)cycloalkyl, (C₁-C₆)alkyl(C₃-C₇)heterocycloalkyl, (C₁-C₆)alkyl-aryl, and (C₁-C₆)alkyl-heteroaryl.

Representative examples included are:
(Endo)-3-(2-methoxy-2,2-di-thiophen-2-yl-ethyl)-8,8-dimethyl-8-azonia-bicyclo[3.2.1]octane iodide;
3-((Endo)-8-methyl-8-aza-bicyclo[3.2.1]oct-3-yl)-2,2-diphenyl-propionitrile;
(Endo)-8-methyl-3-(2,2,2-triphenyl-ethyl)-8-aza-bicyclo[3.2.1]octane;
3-((Endo)-8-methyl-8-aza-bicyclo[3.2.1]oct-3-yl)-2,2-diphenyl-propionamide;
3-((Endo)-8-methyl-8-aza-bicyclo[3.2.1]oct-3-yl)-2,2-diphenyl-propionic acid;
(Endo)-3-(2-cyano-2,2-diphenyl-ethyl)-8,8-dimethyl-8-azonia-bicyclo[3.2.1]octane iodide;
(Endo)-3-(2-cyano-2,2-diphenyl-ethyl)-8,8-dimethyl-8-azonia-bicyclo[3.2.1]octane bromide;
3-((Endo)-8-methyl-8-aza-bicyclo[3.2.1]oct-3-yl)-2,2-diphenyl-propan-1-ol;
N-Benzyl-3-((endo)-8-methyl-8-aza-bicyclo[3.2.1]oct-3-yl)-2,2-diphenyl-propionamide;
(Endo)-3-(2-carbamoyl-2,2-diphenyl-ethyl)-8,8-dimethyl-8-azonia-bicyclo[3.2.1]octane iodide;
1-Benzyl-3-[3-((endo)-8-methyl-8-aza-bicyclo[3.2.1]oct-3-yl)-2,2-diphenyl-propyl]-urea;
1-Ethyl-3-[3-((endo)-8-methyl-8-aza-bicyclo[3.2.1]oct-3-yl)-2,2-diphenyl-propyl]-urea;
N-[3-((Endo)-8-methyl-8-aza-bicyclo[3.2.1]oct-3-yl)-2,2-diphenyl-propyl]-acetamide;
N-[3-((Endo)-8-methyl-8-aza-bicyclo[3.2.1]oct-3-yl)-2,2-diphenyl-propyl]-benzamide;
3-((Endo)-8-methyl-8-aza-bicyclo[3.2.1]oct-3-yl)-2,2-di-thiophen-2-yl-propionitrile;
(Endo)-3-(2-cyano-2,2-di-thiophen-2-yl-ethyl)-8,8-dimethyl-8-azonia-bicyclo[3.2.1]octane iodide;
N-[3-((Endo)-8-methyl-8-aza-bicyclo[3.2.1]oct-3-yl)-2,2-diphenyl-propyl]-benzenesulfonamide;
[3-((Endo)-8-methyl-8-aza-bicyclo[3.2.1]oct-3-yl)-2,2-diphenyl-propyl]-urea;
N-[3-((Endo)-8-methyl-8-aza-bicyclo[3.2.1]oct-3-yl)-2,2-diphenyl-propyl]-methanesulfonamide; and/or
(Endo)-3-{2,2-diphenyl-3-[(1-phenyl-methanoyl)-amino]-propyl}-8,8-dimethyl-8-azonia-bicyclo[3.2.1]octane bromide.

Preferred compounds useful in the present invention include:
(Endo)-3-(2-methoxy-2,2-di-thiophen-2-yl-ethyl)-8,8-dimethyl-8-azonia-bicyclo[3.2.1]octane iodide;
(Endo)-3-(2-cyano-2,2-diphenyl-ethyl)-8,8-dimethyl-8-azonia-bicyclo[3.2.1]octane iodide;
(Endo)-3-(2-cyano-2,2-diphenyl-ethyl)-8,8-dimethyl-8-azonia-bicyclo[3.2.1]octane bromide;
(Endo)-3-(2-carbamoyl-2,2-diphenyl-ethyl)-8,8-dimethyl-8-azonia-bicyclo[3.2.1]octane iodide;
(Endo)-3-(2-cyano-2,2-di-thiophen-2-yl-ethyl)-8,8-dimethyl-8-azonia-bicyclo[3.2.1]octane iodide; and/or
(Endo)-3-{2,2-diphenyl-3-[(1-phenyl-methanoyl)-amino]-propyl}-8,8-dimethyl-8-azonia-bicyclo[3.2.1]octane bromide.

Suitable antihistamines (also referred to as H₁-receptor antagonists) include any one or more of the numerous antagonists known which inhibit H₁-receptors, and are safe for human use. All are reversible, competitive inhibitors of the interaction of histamine with H₁-receptors. The majority of these inhibitors, mostly first generation antagonists, have a core structure, which can be represented by the following formula:

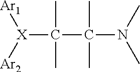

This generalized structure represents three types of antihistamines generally available: ethanolamines, ethylenediamines, and alkylamines. In addition, other first generation antihistamines include those which can be characterized as based on piperizine and phenothiazines. Second generation antagonists, which are non-sedating, have a similar structure-activity relationship in that they retain the core ethylene group (the alkylamines) or mimic the tertiary amine group with piperizine or piperidine. Exemplary antagonists are as follows:
Ethanolamines: carbinoxamine maleate, clemastine fumarate, diphenylhydramine hydrochloride, and dimenhydrinate.
Ethylenediamines: pyrilamine amleate, tripelennamine HCl, and tripelennamine citrate.
Alkylamines: chloropheniramine and its salts such as the maleate salt, and acrivastine.
Piperazines: hydroxyzine HCl, hydroxyzine pamoate, cyclizine HCl, cyclizine lactate, meclizine HCl, and cetirizine HCl.
Piperidines: Astemizole, levocabastine HCl, loratadine or its descarboethoxy analogue, and terfenadine and fexofenadine hydrochloride or another pharmaceutically acceptable salt.

Azelastine hydrochloride is yet another H₁ receptor antagonist which may be used in combination with a PDE4 inhibitor.

The aerosol composition suitably has a concentration of a drug in an amount of from about 0.005 mg/g formulation up to about 1000 mg/g of formulation, for example, 0.005, 0.05, 0.5, 1, 5, 10, 25, 50, 100, 200, 400, 600, 800, 1000 mg/g. Appropriate doses of known therapeutic agents will be readily appreciated by those skilled in the art.

The combinations referred to above may conveniently be presented for use in the form of a pharmaceutical formulation, and thus pharmaceutical formulations comprising combinations of drugs in the aggregate particles represent a further aspect of the invention. It is understood that the aggregate particles as described herein are suitable for delivery, however, it is also considered within the scope of the invention that the aggregate particles may be blended with a physiologically acceptable excipient, e.g. one or more further diluents, carriers, lubricants, stabilizers, etc., as would be understood by one of skill in the art.

One or more excipient materials may be used to make up the excipient nanoparticles, making up the non-aqueous dispersion and resulting aggregate particles. Suitably excipients useful in the invention include, but are not limited to, amino acids, sugars (sacchrides), poly(aminoacids), stearates, sugar fatty acid esters, sugar alcohols, sugar acids, cholesterol, cyclodextrins, EDTA, vitamin E and its derivatives, di-keto piperazine, taste masking agents, and inorganic materials, and any combination thereof.

Particularly preferred excipients include but are not limited to amino acids, such as, leucine, iso-leucine, valine, and glycine; polyamino acids, such as trileucine; sugars, such as lactose, sucrose, glucose and trehalose; synthetic sugars, such as sucralose; sugar alcohols, such as mannitol, sorbitol, inositol, xylitol, erythritol, lactitol, and malitol; sugar acids, such as ascorbic acid; taste masking agents, such as aspartame; stearates, such as magnesium stearate, calcium stearate and sodium stearate; vitamin E derivatives, such as Tocopherols, such as alpha-Tocopherol, gama-Tocopherol, and Tocotrienols; salts, such as sodium chloride and calcium chloride; inorganic carbonates, such as potassium carbonate, calcium carbonate, magnesium carbonate, and ammonium carbonate; inorganic phosphates, such as sodium phosphate, potassium phosphate and calcium phosphate; cyclodextrins; EDTA; and di-keto-piperazine; and citric acid and its salts.

Excipients may be employed in nanoparticulate form either singly, or in any combination. For example, aggregate particles may contain nanoparticulate excipient particles of sugars, such as lactose, and one or more nanoparticlute amino acids, such as nanoparticulate leucine.

A suitable material which may be employed in the composite particles of the present invention as a binder include leucine. Leucine has a desirable level of hydrophobicity and may be incorporated to improve the surface properties of the aggregate particles, thus increasing the dispersability of the aggregate particles from each other. A further advantage of leucine is that if any suspended material is solubilized, the dissolved portion can be spray-dried as crystalline material.

Optionally, a binder may be employed to assist in the formation of the aggregate particles from the nanoparticulate drug and excipient particles.

When employed, the optional binder in the aggregate particles may include one or more polymers, dextrans or substituted dextrans, lipids, and/or surfactants, or the binder may also comprise a quantity of the excipient of the excipient nanoparticles which dissolves in the non-aqueous liquid prior to aggregate formation.

Particularly preferred excipients include but are not limited to polymeric binders, such s PLGA (poly(lactic-co-glycolic acid)), PLA (Poly(lactic acid) or polylactide), PEG (Polyethylene glycol), chitosan, PVP (Polyvinylpyrrolidone), PVA (Polyvinyl alcohol), and hyaluronic acid; lipid binders, such as DPPC (Dipalmitoylphosphatidylcholine) and DSPC (,2-distearoly-sn-glycero-3-phosphocholine), and/or other lung surfactants; non-ionic surfactants, such as sorbitan esters, such as Sorbitane trioleate (Span85); anionic surfactant, sodium laurel sulfate; polysorbates, such as polyethylene glycol sorbitan monolaurate (Tween20), alone or in any combination. One or more binders may be employed in the agglegate particles.

The binder may also play a role in imparting certain characteristics upon the aggregate particle. For example, the aggregates of the present inventions may employ binder materials which are endogenous to the lung, such as DPPC or lecithin, which are approved as generally accepted as safe (“GRAS”). Since they are endogenous to the lung, these materials have the potential to not be perceived as being foreign. As such the body would not mount a macrophage response, due to their presence. Further, by carefully selecting binder materials, it may be possible to alter the dissolution rate of the active therapeutic ingredient(s), potentially affecting the pharmacokinetic and pharmacodynamic (PK/PD) characteristics of the composition.

The binder may also assist in defining a stable and chemically uniform surface. Thus, the aerosol composition may be made with very predictable performance and powder flow characteristics, as the binder may dominate the external physical characteristics and, correspondingly, the physical stability of the composite particles.

Binder, when incorporated into the aggregate particles, makes up from 0.1 to 30% of the composition of the aggregate particles. Preferably, the binder is 20% or less, such as 15, 10, 5, 2.5, or 1% of the composition of the aggregate particles.

In further embodiemnts, the present invention relates to a method of making aggregate particles suitable for a powder aerosol composition comprising:
(a) forming a dispersion of nanoparticulate drug particles and/or nanoparticulate excipient particles in a non-aqueous liquid,
   wherein said drug particles and/or said excipient particles have a solubility of less than 10 mg/ml in said liquid dispersing media, and
   wherein the nanoparticulate drug particles have a preselected crystalline form,
   and wherein the non-aqueous liquid has no suspension homogenizing surfactant dissolved therein;
(b) spray-drying the dispersion of nanoparticulate drug particles and/or nanoparticulate excipient particles to generate aggregate particles comprising nanoparticulate drug particles and/or nanoparticulate excipient particles,
   wherein the drug and/or excipient nanoparticles have maintained their preselected crystalline form, and
   wherein the aggregate particles have a mass median aerodynamic diameter of less than or equal to about 100 microns
   and wherein the aggregate particles is substantially free of a homogenizing surfactant.

A still further aspect of the present invention relates to a method of making a dry powder aerosol composition comprising:
(a) forming, in a non-aqueous liquid, a dispersion of nanoparticulate drug particles and nanoparticulate excipient particles,
(b) spray-drying the dispersion of nanoparticulate drug particles and nanoparticulate excipient particles to form a dry powder of aggregates of the nanoparticulate drug particles and nanoparticulate excipient particles, wherein the aggregates comprise both drug and excipient particles and have a diameter of less than or equal to about 100 microns.

In certain embodiments the method includes the step of including binder in the nanoparticulate non-aqueous dispersion prior to spray-drying, wherein following spray-drying, essentially every aggregate contains one or more nanoparticulate drug particle, one or more nanoparticulate excipient nanoparticles and binder.

Suitably, the binder is dissolved in the liquid phase of the non-aqueous dispersion. The non-aqueous liquid in which the drug and excipient particles are dispersed prior to drying (and/or during nanoparticles creation) can be any non-aqueous media desired, having appropriate characteristics for its intended use, as would be readily determinable by those of ordinary skill. Suitable non-aqueous dispersing media include, but are not limited to alcohols, ketones, esters, (cyclic or linear) alkanes, ethers, chlorinated hydrocarbons, fluorinated hydrocarbons, and mixtures of thereof. Suitability of a given non-aqueous liquid will be influence by the nanoparticulate drug and nanoparticulate excipient selected. As mentioned above, the nanoparticulate drug particles and nanoparticulate excipient particles are suitably “poorly soluble” in the non-aqueous dispersing media, having a solubility in the non-aqueous liquid of less than about 10 mg/ml.

Examples of suitable non-aqueous liquid media include, but are not limited to, alcohols, such as ethanol and propanol; ketones, such as acetone and methylethylketone; esters, such as ethyl acetate and isopropylacetate; linear alkanes, such as isooctane, cyclic oalkanes, such as cyclohexane and methylcyclohexane; ethers, such as methyl-tert-butyl ether and cyclopentylmethylether; chlorinated hydrocarbons, such as p11 (Fluorotrichloromethane) and p12 (Difluorodichloromethane); fluorinated hydrocarbons, such as include p134a (1,1,1,2-Tetrafluoroethane) and p227 (1,1,1,2,3,3,3-Heptafluoropropane); or any combination thereof.

Aggregate particle production is preferably achieved by spray drying, wherein the aggregate particles comprising nanoparticulate drug and nanoparticulate excipient may be prepared from non-aqueous liquid suspension feedstock which contains the nanoparticulate drug material. Suitable spray driers include the Niro Mobile Minor and PSD-1 spray driers. Co-current and mixed flow drying configurations may be employed. Thus, a Niro Pharmaceutical Spray Drier, Model PSD-1, equipped with an operable peristaltic a Watson Marlow pump 505 may be employed for such purposes. The spray drier may be fitted with a suitable spray nozzle, such as a Spraying Systems two-fluid SU-4 60/100 with 120 cap, or a rotary nozzle.

With the spray nozzle, a two fluid nozzle may employ nitrogen as an atomizing gas. Suitable inlet temperatures for this purpose are also between 80 to 180 degrees Celsius. Other inlet temperatures may be used depending on the physicochemical properties of the non-aqueous feedstock and the feedstock feed rate.

The suspension feedstock may be supplied at a desired feed rate, and the inlet temperatures set as desired. Exemplary feed rates are 30 to 120 mL/min. Rotary nozzles may be operated at up to 35000 RPM.

Nitrogen may also be used as both atomizing gas and the drying gas.

Spray dried powders may be collected using a cyclone or bag filter at the drier outlet.

The feedstock for spray drying may be of the nanoparticulate material, alone or in combination with further excipients which are presented in solution, including such materials as binders.

As will be understood, the non-aqueous feedstock may contain a single type of nanoparticulate drug, or more than one type nanoparticulate drug, combined with one or more nanoparticulate excipients, optionally with a binder. Such an approach yields a combination of drugs combined in each aggregate particle.

In further embodiments, the feedstock contains nanoparticles of one of more nanoparticulate drugs and nanoparticles of one or more excipients. The drug nanoparticles and excipient nanoparticles may be included in the same feed stock as a result of co-milling, as described previously, or may they have been milled separately and combined/admixed prior to spray drying. Similarly, some nanoparticles materials may be co-milled in a single suspension, while others were created independently, and these various suspensions subsequently combined/admixed prior to aggregate production.

The drug and excipient feedstock(s) may be fed into the spray drier with or without binder material.

Size of individual particles may be determined by scanning electron microscope (SEM).

The aggregate particles described herein may be delivered by any suitable delivery system.

Combinations described herein are preferably administered either sequentially or simultaneously in separate or combined pharmaceutical formulations. Devices for accomplishing such delivery are known in the art.

In certain embodiments, the combination of different actives in the same aggregate particle may be readily achieved by the approach herein described, and the inclusion of multiple therapeutically active materials in the same aggregate particle not only enables delivery of combination therapies, but also assures co-deposition of both actives in the same targeted location and, perhaps to the same cell. Thus, use of multiple active pharmaceutical ingredients in a single composite particle may facilitate synergistic effects within cells.

Aerosols can be defined as colloidal systems consisting of very finely divided dry powder particles dispersed in or surrounded by a gas. Formulation of the aggregate particles of the present invention thus facilitate dispersion of the aggregate particles into this colloidal state.

Desirably, the formulation and delivery system employed maximizes the percentage of the formulation which exits the delivery device, and maximizes the percentage of the aggregate particles which exits the device being delivered to the target region of the body.

The powders are deliverable from suitable delivery systems for entraining powders, including for example, dry powder inhalers (DPIs) or metered dose inhalers (MDIs) as aerosols.

One embodiment of the present invention comprises an aerosol dosage form comprising the dry powder aggregate particles comprising nanoparticulate drug particles, nanoparticulate excipient particles, and optionally binder.

In a further embodiment of the invention, the aggregate particles may be formulated as a dry powder formulation for use in a dry powder inhalation device, and are admixed with physiologically acceptable carrier or diluent. While any suitable carrier or diluent excipient material, or blend of materials, may be used. In one suitable embodiment, the excipient carrier or diluent particles are lactose, mannitol or starch. Advantageously, such admixed formulation may possess beneficially enhanced delivery and dispersion efficiencies. This approach also may be used to further dilute high potency drugs, or where further diluents are desirable to allow for metering and/or dose adjustment.

Where the dry powder aerosol composition of the invention is used in a propellant-based pressurized MDI, the powder composition is formulated with a pressurized non-aqueous propellant. In certain preferred embodiments of the invention, the propellant use formulation is in a less ozone depleting, more environmentally friendly, non-CFC propellant, such as p134a (1,1,1,2-Tetrafluoroethane) or p227 (1,1,1,2,3,3,3-Heptafluoropropane). The propellant formulation may also include one or more solvents, co-solvents, surfactants, etc., as will be appreciated by those skilled in MDI formulation. In the case of a pressurized aerosol the dosage unit may be determined by providing a valve to deliver a metered amount.

Capsules and cartridges of e.g. gelatin for use in an inhaler or insufflator may be formulated containing a powder mix of a compound of the invention and a suitable powder base such as lactose or mannitol.

Suitably, in one embodiment of the present invention, the aggregates of the present invention are delivered via oral inhalation or intranasal administration. Appropriate dosage forms for such administration, such as an aerosol formulation or a metered dose inhaler, may be prepared by conventional techniques.

The invention also relates to a dry powder aerosol composition for use in a DPI.

Dry powder compositions for topical or systemic delivery to the lung by inhalation may, for example, be presented in capsules and cartridges of for example gelatin or blisters of for example laminated aluminum foil, for use in an inhaler or insufflator. Powder blend formulations generally contain a powder mix for inhalation of the aggregate particles of the invention alone, or with a suitable powder base (carrier/diluent/excipient substance) such as mono-, di or poly-saccharides (e.g. lactose or starch). Use of lactose is preferred.

Each capsule or cartridge may generally contain one or more therapeutically active compound. With a single type of active compound, a single aggregate particle type will be in the composition. With multiple actives, the composition will contain a single aggregate type which contains more than one type of therapeutic agent, or, alternatively, the composition will contain multiple aggregate types with each aggregate type containing separate active agent(s), which are admixed in the composition.

The aggregate particles may comprise one or more active nanoparticulate ingredients. Further, the aggregates may be blended with non-active containing excipient, which may be desirable, especially for extremely potent therapeutically active material(s).

Suitably, the packing/medicament dispenser is of a type selected from the group consisting of a DPI (either a reservoir based DPI, a single dose DPI, or a multi-dose DPI), and a metered dose inhaler (MDI).

By reservoir based DPI it is meant an inhaler having a reservoir form pack suitable for comprising multiple (un-metered doses) of medicament in dry powder form and including means for metering medicament dose from the reservoir to a delivery position. The metering means may for example comprise a metering cup, which is movable from a first position where the cup may be filled with medicament from the reservoir to a second position where the metered medicament dose is made available to the patient for inhalation.

By multi-dose dry powder inhaler (MDPI) is meant an inhaler suitable for dispensing medicament in dry powder form, wherein the medicament is comprised within a multi-dose pack containing (or otherwise carrying) multiple, define doses (or parts thereof) of medicament. In a preferred aspect, the carrier has a blister pack form, but it could also, for example, comprise a capsule-based pack form or a carrier onto which medicament has been applied by any suitable process including printing, painting and vacuum occlusion.

In the case of multi-dose delivery, the formulation can be pre-metered (e.g. as in DISKUS®, see GB 2242134, U.S. Pat. Nos. 6,632,666, 5,860,419, 5,873,360 and 5,590,645 or DISKHALER®, see GB 2178965, 2129691 and 2169265, U.S. Pat. Nos. 4,778,054, 4,811,731, 5,035,237, the disclosures of which are hereby incorporated by reference) or metered in use (e.g. as in TURBUHALER®, see EP 69715 or in the devices described in U.S. Pat. No. 6,321,747 the disclosures of which are hereby incorporated by reference). An example of a unit-dose device is ROTAHALER® (see GB 2064336 and U.S. Pat. No. 4,353,656, the disclosures of which are hereby incorporated by reference).

The DISKUS® inhalation device comprises an elongate strip formed from a base sheet having a plurality of recesses spaced along its length and a lid sheet hermetically but peelably sealed thereto to define a plurality of containers, each container having therein an inhalable formulation containing a compound of formula (I) or (Ia) preferably combined with lactose. Preferably, the strip is sufficiently flexible to be wound into a roll. The lid sheet and base sheet will preferably have leading end portions which are not sealed to one another and at least one of the said leading end portions is constructed to be attached to a winding means. Also, preferably the hermetic seal between the base and lid sheets extends over their whole width. The lid sheet may preferably be peeled from the base sheet in a longitudinal direction from a first end of the said base sheet.

In one embodiment, the multi-dose pack is a blister pack comprising multiple blisters for containment of medicament in dry powder form. The blisters are typically arranged in regular fashion for ease of release of medicament there from.

In a further embodiment of the invention, the multi-dose blister pack comprises plural blisters arranged in generally circular fashion on a disc-form blister pack. In another aspect, the multi-dose blister pack is elongate in form, for example comprising a strip or a tape.

In a further embodiment of the present invention, the multi-dose blister pack is defined between two members peelably secured to one another. U.S. Pat. Nos. 5,860,419, 5,873,360 and 5,590,645 describe medicament packs of this general type. In this aspect, the device is usually provided with an opening station comprising peeling means for peeling the members apart to access each medicament dose. Suitably, the device is adapted for use where the peelable members are elongate sheets which define a plurality of medicament containers spaced along the length thereof, the device being provided with indexing means for indexing each container in turn. More preferably, the device is adapted for use where one of the sheets is a base sheet having a plurality of pockets therein, and the other of the sheets is a lid sheet, each pocket and the adjacent part of the lid sheet defining a respective one of the containers, the device comprising driving means for pulling the lid sheet and base sheet apart at the opening station.

By metered dose inhaler (MDI) it is meant a medicament dispenser suitable for dispensing medicament in aerosol form, wherein the medicament is comprised in an aerosol container suitable for containing a propellant-based aerosol medicament formulation. The aerosol container is typically provided with a metering valve, for example a slide valve, for release of the aerosol form medicament formulation to the patient. The aerosol container is generally designed to deliver a predetermined dose of medicament upon each actuation by means of the valve, which can be opened either by depressing the valve while the container is held stationary or by depressing the container while the valve is held stationary.

Where the medicament container is an aerosol container, the valve typically comprises a valve body having an inlet port through which a medicament aerosol formulation may enter said valve body, an outlet port through which the aerosol may exit the valve body and an open/close mechanism by means of which flow through said outlet port is controllable.

The valve may be a slide valve wherein the open/close mechanism comprises a sealing ring and receivable by the sealing ring a valve stem having a dispensing passage, the valve stem being slidably movable within the ring from a valve-closed to a valve-open position in which the interior of the valve body is in communication with the exterior of the valve body via the dispensing passage.

Typically, the valve is a metering valve. The metering volumes are typically from 10 to 100 μl, such as 25 μl, 50 μl or 63 μl. Suitably, the valve body defines a metering chamber for metering an amount of medicament formulation and an open/close mechanism by means of which the flow through the inlet port to the metering chamber is controllable. Preferably, the valve body has a sampling chamber in communication with the metering chamber via a second inlet port, said inlet port being controllable by means of an open/close mechanism thereby regulating the flow of medicament formulation into the metering chamber.

The valve may also comprise a ‘free flow aerosol valve’ having a chamber and a valve stem extending into the chamber and movable relative to the chamber between dispensing and non-dispensing positions. The valve stem has a configuration and the chamber has an internal configuration such that a metered volume is defined there between and such that during movement between is non-dispensing and dispensing positions the valve stem sequentially: (i) allows free flow of aerosol formulation into the chamber, (ii) defines a closed metered volume for pressurized aerosol formulation between the external surface of the valve stem and internal surface of the chamber, and (iii) moves with the closed metered volume within the chamber without decreasing the volume of the closed metered volume until the metered volume communicates with an outlet passage thereby allowing dispensing of the metered volume of pressurized aerosol formulation. A valve of this type is described in U.S. Pat. No. 5,772,085.

Additionally, intra-nasal delivery of the present compounds is effective.

To formulate an effective pharmaceutical nasal composition, the medicament must be delivered readily to all portions of the nasal cavities (the target tissues) where it performs its pharmacological function. Additionally, the medicament should remain in contact with the target tissues for relatively long periods of time. The longer the medicament remains in contact with the target tissues, the medicament must be capable of resisting those forces in the nasal passages that function to remove particles from the nose. Such forces, referred to as ‘mucociliary clearance’, are recognized as being extremely effective in removing particles from the nose in a rapid manner, for example, within 10-30 minutes from the time the particles enter the nose.

Other desired characteristics of a nasal composition are that it must not contain ingredients which cause the user discomfort, that it has satisfactory stability and shelf-life properties, and that it does not include constituents that are considered to be detrimental to the environment, for example ozone depletors.

A suitable dosing regimen for the formulation of the present invention when administered to the nose would be for the patient to inhale deeply subsequent to the nasal cavity being cleared. During inhalation the formulation would be applied to one nostril while the other is manually compressed. This procedure would then be repeated for the other nostril.

MDI aerosol compositions suitable for inhalation are suspension based generally contain the aggregates of the present invention, optionally in combination with another therapeutically active ingredient, and a suitable propellant such as a fluorocarbon or hydrogen-containing chlorofluorocarbon or mixtures thereof, particularly hydrofluoroalkanes, e.g. dichlorodifluoromethane, trichlorofluoromethane, dichlorotetrafluoroethane, especially 1,1,1,2-tetrafluoroethane, 1,1,1,2,3,3,3-heptafluoro-n-propane or a mixture thereof. Carbon dioxide or other suitable gas may also be used as propellant. The aerosol composition may be excipient free or may optionally contain additional formulation excipients well known in the art such as surfactants, e.g., oleic acid or lecithin and cosolvents, e.g. ethanol. Pressurised formulations will generally be retained in a canister (e.g. an aluminium canister) closed with a valve (e.g. a metering valve) and fitted into an actuator provided with a mouthpiece.

Medicaments for administration by inhalation desirably have a controlled particle size. The desired fraction may be separated out by air classification or sieving.

For all methods of use disclosed herein the daily inhalation dosage regimen will depend upon the drug or drugs being delivered, and preferably be from about 40 mg to 0.5 mcg/day; such as 1 milligram to 10 mcg per day, such as 500 to 50 mcg per day, administered in one or more daily doses.

It will also be recognized by one of skill in the art that the optimal quantity and spacing of individual dosages of drug will be determined by the nature and extent of the condition being treated, the form, route and site of administration, and the particular patient being treated, and that such optimums can be determined by conventional techniques. It will also be appreciated by one of skill in the art that the optimal course of treatment, i.e., the number of doses given per day for a defined number of days, can be ascertained by those skilled in the art using conventional course of treatment determination tests.

The aggregates of nanoparticulate drug and nanoparticulate excipient of the present invention may also be used in association with the veterinary treatment of mammals, other than humans, in need thereof.

For use herein treatment may include prophylaxis for use in a treatment group susceptible to such infections. It may also include reducing the symptoms of, ameliorating the symptoms of, reducing the severity of, reducing the incidence of, or any other change in the condition of the patient, which improves the therapeutic outcome.

It should be understood that in addition to the ingredients particularly mentioned above, the formulations of this invention may include other agents conventional in the art having regard to the type of formulation in question, for example, for inhalation may or may not include further carrier particles, such as lactose.

### METHODS AND EXAMPLES

The following examples are given to illustrate the present invention. It should be understood, however, that the invention is not to be limited to the specific conditions or details described in these examples.

### Preparation of Samples

The formulations listed in Table 1 were manufactured using a non-aqueous bead milling process followed by spray drying. A Drais Cosmo 2 Mill was used. The mill was setup using the parameters listed in Table 2. Drug and excipients were weighed into a suitable container. Non-aqueous liquid media was added to the container and the contents shaken until all powder was visibly wetted. The suspension was poured into the mill reservoir where ˜200 mL of the non-aqueous liquid vehicle was already re-circulating. The suspension was milled for the desired duration, and then collected. The suspension was stored in a closed container at ambient conditions until spray drying was performed.

For those Samples using a binder which was not a dissolved portion of the drug nanoparticles and/or excipient nanoparticles, a solution of binder was admixed with the nanoparticulate suspension to achieve the desired concentration of binder shown in Table 1, prior to spray drying,

Spray drying was performed using a Niro PSD-1 dryer. Table 3 lists the dryer parameters used. Spray drying is advantageously suitable for fabricating such aggregate particles. The size of the aggregates can be controlled by the spray drying conditions, independent of the drug and excipient. The flexibility and control associated with spray drying, may allow production of particles with desirable aerodynamic properties, thus permitting high efficiency delivery of medicaments.

Powder was collected in a container beneath the cyclone and subsequently harvested in a low humidity chamber.

TABLE 1
Examples of Powder Formulations

| | | | | Excipient | | Excipient | | |
|---|---|---|---|---|---|---|---|---|
| | Manufacture | API | Excipient | 1 | Excipient | 2 | | Binder |
| Sample | Method | % w/w | 1 | % w/w | 2 | % w/w | Binder | % w/w |
| 1 | Bead milled | 100% API-A | — | — | — | — | — | — |
| 2 | Co-milled | 50% API-A | Lactose | 50 | — | — | — | — |
| 3 | Co-milled | 5% API-A | Lactose | 95 | — | — | — | — |
| 4 | Co-milled | 5% API-A | Mannitol | 95 | — | — | — | — |
| 5 | Co-milled | 90% API-B | Magnesium | 10 | — | — | — | — |
| | | | stearate | | | | | |
| 6 | Co-milled | 90% API-A | Leucine | 10 | — | — | — | — |
| 7 | Co-milled | 50% API-A | Leucine | 50 | — | — | — | — |
| 8 | Admixed | 50% API-A | Leucine | 50 | — | — | — | — |
| 9 | Co-milled | 5% API-A | Leucine | 95 | — | — | — | — |
| 10 | Bead milled | 90% API-A | — | — | — | — | Lecithin | 10 |
| 11 | Bead milled | 90% API-A | — | — | — | — | DPPC | 10 |
| 12 | Bead milled | 85% API-A | — | — | — | — | DPPC | 15 |
| 13 | Bead milled | 80% API-A | — | — | — | — | DPPC | 20 |
| 14 | Co-milled | 45% API-A | Lactose | 45 | — | — | DPPC | 10 |
| 15 | Co-milled | 5% API-A | Lactose | 85 | — | — | DPPC | 10 |
| 16 | Admixed | 5% API-A | Lactose | 85 | — | — | DPPC | 10 |
| 17 | Co-milled | 5% API-A | Mannitol | 85 | — | — | DPPC | 10 |
| 18 | Co-milled | 45% API-A | Lactose | 45 | Leucine | 10 | — | — |
| 19 | Co-milled | 5% API-A | Lactose | 85 | Leucine | 10 | — | — |
| 20 | Co-milled | 5% API-A | Lactose | 85 | Leucine | 10 | — | — |
| 21 | Co-milled | 5% API-A | Mannitol | 85 | Leucine | 10 | — | — |

TABLE 2
Mill Parameters

| | |
|---|---|
| Grinding Media | 0.3 mm yttrium stabilized zirconium oxide grinding |
| | beads |
| Configuration | Suspension re-circulated between reservoir and |
| | milling chamber using the integral peristaltic pump |
| | Pneumatic stirrer used to mix contents of reservoir |
| | during processing |
| Sieve Screen | 0.15 mm |
| Mill Speed (%) | 80 |
| Recirculating | 9 |
| Pump Speed | |
| Setting | |

TABLE 3
Spray Dryer Parameters

| | |
|---|---|
| Dryer | Niro Pharmaceutical Spray Dryer (Model |
| | PSD-1) |
| Feed pump | Watson Marlow pump 505L |
| Collection method | High efficiency cyclone |
| Drying Nitrogen Flow Rate | 75 |
| (kg/hr) | |
| Inlet Temperature | 180 |
| (Celsius) | |
| Outlet Temperature | 95 |
| (Celsius) | |
| Atomizer | Two fluid nozzle: Spraying Systems Co., |
| | Top spray 60/100 with 120 cap |
| Atomization Pressure (psi) | 50.75 |
| Suspension feed rate | 60 |
| (mL/min) | |

TABLE 4
Suspension Feedstocks Used to Prepare Formulation Powders

| | | Approx. | Target Quantities | | | | |
|---|---|---|---|---|---|---|---|
| | | Suspension | | Excipient | Excipient | | Approx. |
| | Suspension | Concentration | API base | 1 | 2 | Vehicle | Milling Time |
| Sample | Feedstock | (% w/v) | (g) | (g) | (g) | (mL) | (hours) |
| 1 | 7.5% w/v API-A in Ethyl Acetate | 7.5 | 97.5 | — | — | 1300 | 5 |
| 2 | 3.75% w/v API-A + 3.75% w/v | 7.5 | 48.75 | 48.75 | — | 1300 | 2.5 |
| | Lactose in Ethyl Acetate | | | | | | |
| 3 | 0.25% w/v API-A + 4.75% w/v | 5 | 3.3 | 61.8 | — | 1300 | 2.5 |
| | Lactose in Ethyl Acetate | | | | | | |
| 4 | 0.25% w/v API-A + 4.75% w/v | 5 | 3.3 | 61.8 | — | 1300 | 2.5 |
| | Mannitol in Ethyl Acetate | | | | | | |
| 5 | 4.5% w/v API-B + 0.5% w/v | 5 | 58.5 | 6.5 | — | 1300 | 2.5 |
| | MgSt in isooctane | | | | | | |
| 6 | 6.75% w/v API-A + 0.75% w/v | 7.5 | 87.8 | 9.8 | — | 1300 | 3.5 |
| | Leucine in Ethyl Acetate | | | | | | |
| 7 | 3.75% w/v API-A + 3.75% w/v | 7.5 | 22.5 | 22.5 | — | 600 | 3 |
| | Leucine in Isopropyl Acetate | | | | | | |
| 8 | 7.5% w/v API-A in Isopropyl Acetate | 7.5 | 97.5 | — | — | 1300 | 2.5 |
| | 7.5% w/v Leucine in Isopropyl acetate | 7.5 | — | 97.5 | — | 1300 | 2.5 |
| 9 | 0.25% w/v API-A + 4.75% w/v | 5 | 3.3 | 61.8 | — | 1300 | 2.5 |
| | Leucine in Ethyl Acetate | | | | | | |
| 10 | 7.5% w/v API-A in Ethyl Acetate | 7.5 | 97.5 | — | — | 1300 | 3 |
| | Sufficient 1.7% w/v Lecithin in | | | | | | |
| | Ethyl Acetate added before spray | | | | | | |
| | drying for binder | | | | | | |
| 11 | 7.5% w/v API-A in Ethyl Acetate | 7.5 | 97.5 | — | — | 1300 | 2.5 |
| | Sufficient 3% w/v DPPC in Ethanol | | | | | | |
| | added before spray drying for binder | | | | | | |
| 12 | 7.5% w/v API-A in Ethyl Acetate | 7.5 | 97.5 | — | — | 1300 | 3 |
| | Sufficient 3% w/v DPPC in Ethanol | | | | | | |
| | added before spray drying for binder | | | | | | |
| 13 | 7.5% w/v API-A in Ethyl Acetate | 7.5 | 97.5 | — | — | 1300 | 3 |
| | Sufficient 3% w/v DPPC in Ethanol | | | | | | |
| | added before spray drying for binder | | | | | | |
| 14 | 3.75% w/v API-A + 3.75% w/v | 7.5 | 48.75 | 48.75 | — | 1300 | 2.5 |
| | Lactose in Ethyl Acetate | | | | | | |
| | Sufficient 3% w/v DPPC in Ethanol | | | | | | |
| | added before spray drying for binder | | | | | | |
| 15 | 0.3% w/v API-A + 4.7% w/v | 5 | 4.3 | 61.4 | — | 1300 | 2.5 |
| | Lactose in Ethyl Acetate | | | | | | |
| | Sufficient 3% w/v DPPC in Ethanol | | | | | | |
| | added before spray drying for binder | | | | | | |
| 16 | 7.5% w/v API-A in Ethyl Acetate | 7.5 | 97.5 | — | — | 1300 | 2.5 |
| | 5% w/v Lactose in Ethyl acetate | 5 | — | 65 | — | 1300 | 2.5 |
| | Sufficient 3% w/v DPPC in Ethanol | | | | | | |
| | added before spray drying for binder | | | | | | |
| 17 | 0.3% w/v API-A + 4.7% w/v Mannitol | 5 | 3.6 | 61.4 | — | 1300 | 2.5 |
| | in Ethyl Acetate | | | | | | |
| | Sufficient 3% w/v DPPC in Ethanol | | | | | | |
| | added before spray drying for binder | | | | | | |
| 18 | 3.38% w/v API-A + 3.38% w/v + | 7.5 | 43.9 | 43.9 | 9.75^{a} | 1300 | 2.5 |
| | Lactose 0.75% w/v Leucine | | | | | | |
| | in Ethyl acetate | | | | | | |
| 19 | 0.25% w/v API-A + 4.25% w/v | 5 | 3.25 | 55.25 | 6.5^{a} | 1300 | 2.5 |
| | Lactose + 0.5% w/v Leucine | | | | | | |
| | in ethyl acetate | | | | | | |
| 20 | 0.25% w/v API-A + 4.25% w/v | 5 | 3.25 | 55.25 | 6.5^{a} | 1300 | 2.5 |
| | Lactose + 0.5% w/v Leucine in | | | | | | |
| | Iso-octane | | | | | | |
| 21 | 0.25% w/v API-A + 4.25% w/v | 5 | 3.25 | 55.25 | 6.5^{a} | 1300 | 2.5 |
| | Mannitol + 0.5% w/v Leucine in | | | | | | |
| | Ethyl Acetate | | | | | | |

^{a}Leucine.

### Testing of Samples

The particle size distribution (PSD) of the suspensions were measured by a wet laser diffraction method using a Malvern 2000 Instrument.

The particle size distribution of the powder samples described in Table 1 was measured by dry laser diffraction method using a Sympatec Particle Size Instrument.

The crystallinity and form of the powder samples were measured by X-Ray Powder Diffraction (XRPD).

The aerodynamic performance of the powders described in Table 1 was determined by cascade impaction. Approximately 4 mg of powder was weighed into size 3 HPMC capsules. Capsules were inserted into a Cyclohaler device (Novartis AG) and delivered into either a Next Generation Impactor or a Fast Screening Impactor (FSI) (commercially available from MSP Corp (Shoreview, Minn., USA)) at 60 L/min. Selected formulation capsules were then overwrapped and desiccated and placed on stability at 30 C/65% RH for 1 and 3 months and retested again in the Cyclohaler.

Aerodynamic performance results are listed in Table 5 as percent fine particle dose (% FPD) of the nominal. The values in Table 5 are of “corrected” nominal, which take into account the active pharmaceutical ingredient (API) content of the powder, in addition to the capsule fill weight. The performance of 100% ^{w}/_{w} micronized API and 4% ^{w}/_{w} API in lactose carrier were determined for comparison purposes using the Cyclohaler and under the same test conditions as the samples.

### Materials

L-Leucine was obtained from Sigma Aldrich. Mannitol (Pearlitol 25C©) was obtained from Roquette Inc. These excipients were coarsely ground using a mortar and pestle prior to use in suspension manufacturing. Lactose monohydrate was obtained from Freisland Foods Domo Ltd. Ethyl acetate, isopropyl acetate and iso-octane was obtained from Sigma Aldrich.

The compound biphenyl-2-ylcarbamic acid 1-[2-(2-chloro-4-{[(R)-2-hydroxy-2-(8-hydroxy-2-oxo-1,2-dihydroquinolin-5-yl)ethylamino]methyl}-5-methoxyphenylcarbamoyl)ethyl]piperidin-4-yl ester, which may be prepared, for example as disclosed in Published PCT Application WO 2007/090859, was used a model drug (mentioned as API-A (active pharmaceutical ingredient-A)) for the data show in the Tables below.

The compound, 6α,9α-Difluoro-1,8-hydroxy-16α-methyl-3-oxo-17α-(2,2,3,3-tetramethycyclopropylcarbonyl)oxy-androsta-1,4-diene-176-carboxylic acid cyanomethyl ester, which may be prepared as disclosed, for example in U.S. Pat. No. 7,288,536, was used as a model drug (mentioned as API-B (active pharmaceutical ingredient-B)) for the data show in Samples 5 and 22 in the Tables below.

### Example 1

The purpose of this example was to demonstrate the technique of manufacturing two-component respiratory particles comprised of nanoparticulate drug and nanoparticulate excipient. Samples 2-7 and 9 in Table 1 utilized a co-milling approach, in which both the drug and excipient were milled together in the bead mill to produce the feedstock suspension (Tables 1 and 4).

Sample 8 was prepared by milling the drug and excipient separately. The drug and excipient suspensions were then admixed in a suitable container and well stirred.

Suspensions were diluted down to 5% w/v with vehicle prior to spray drying.

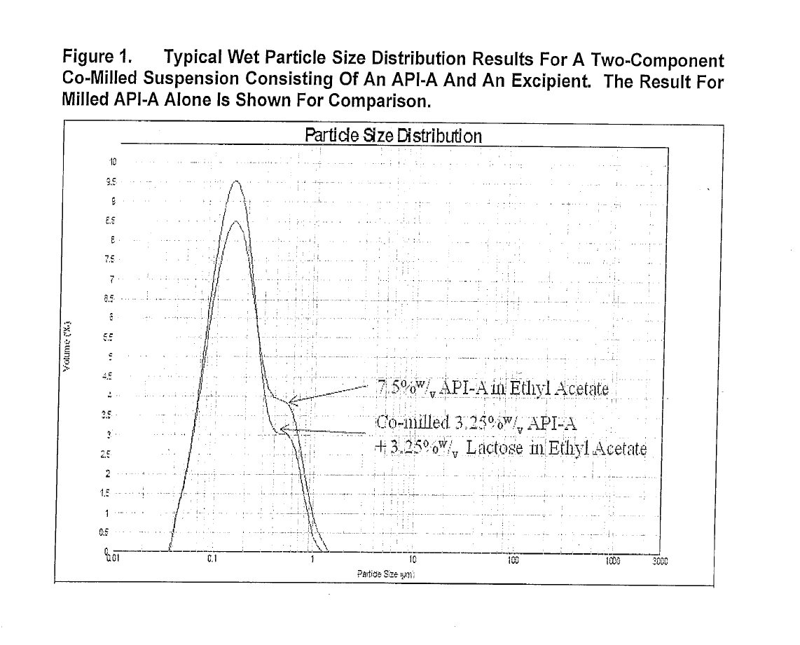

FIG. 1 presents the typical wet PSD results for bead milled API and a two-component suspension system consisting of drug (API) and an excipient. Following bead milling, the majority of suspension particles were less than 1 micron.

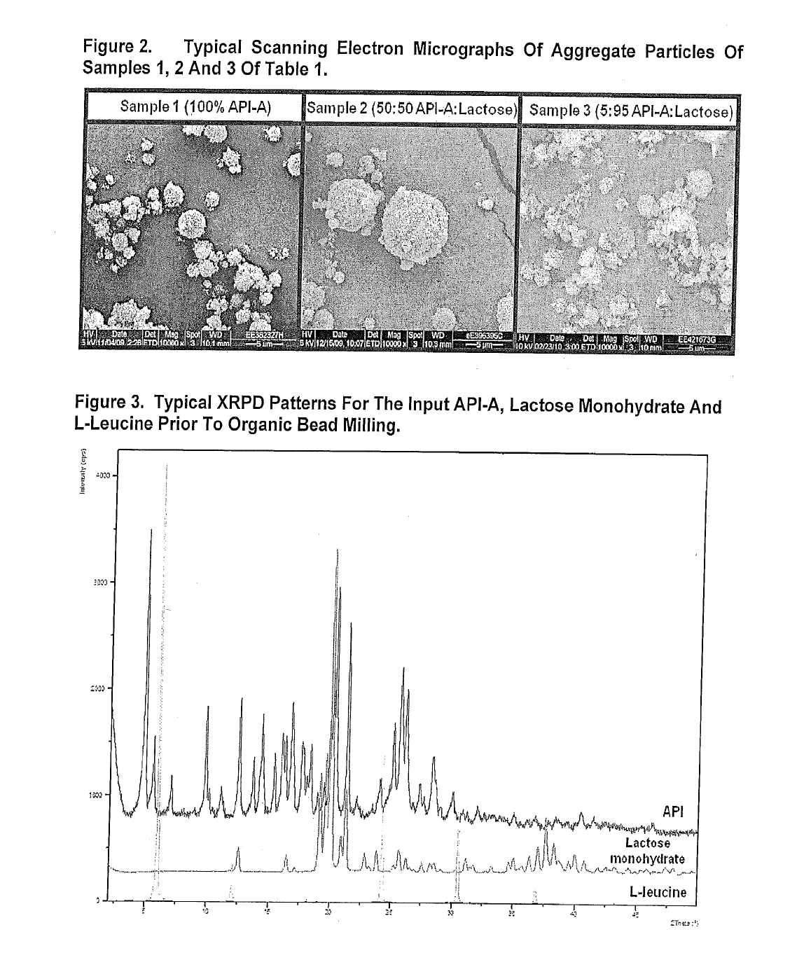

FIG. 2 displays typical SEM micrographs of the spray dried particles. Images of samples 1-3 are displayed. Particles were generally spherical to irregular in shape.

FIG. 3 shows the typical XRPD patterns for the input API, lactose monohydrate and L-leucine prior to organic bead milling.

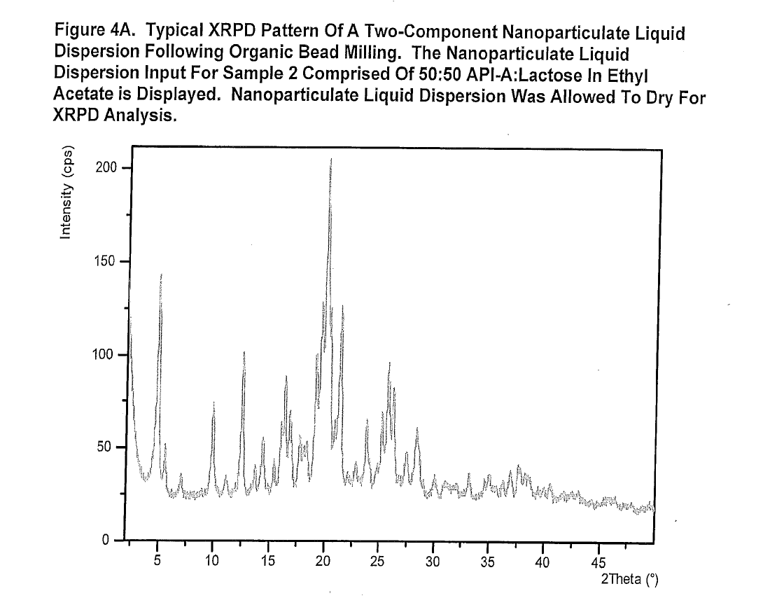

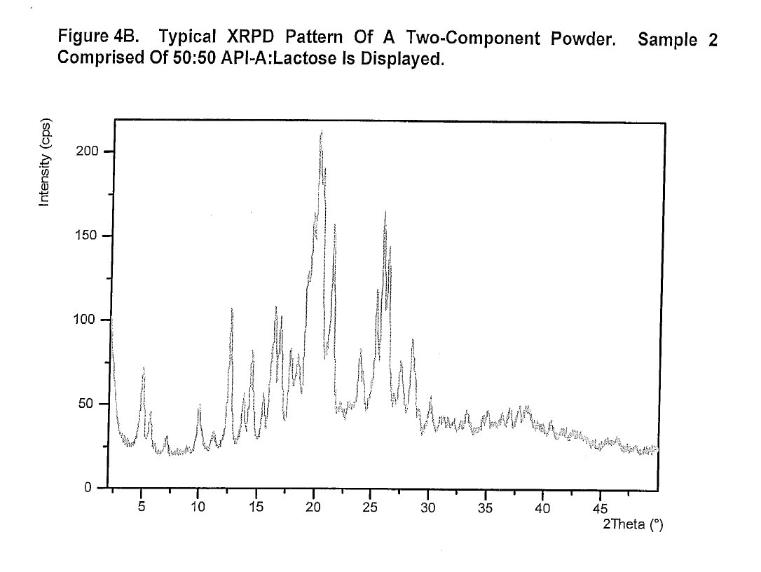

FIG. 4A shows a typical XRPD pattern for a two-component nanoparticulate liquid dispersion following organic bead milling. FIG. 4B shows a typical XRPD pattern for a two-component powder after spray drying. This manufacturing approach maintains the preselected crystallinity of the input powders and produces substantially crystalline product. Table 5 lists the PSD results for samples 2 through 9. The results suggested that the two-component particles were within the respirable size range. Compared to micronized API alone and 4% micronized API blended with lactose carrier, the aerodynamic performance of samples 2 through 9 was improved when delivered by a Cyclohaler into either a NGI or FSI. These results show how API concentration can be flexibly modified by incorporating nanosized excipient, whilst enhancing delivery efficiency.

The present approach benefits from maintaining a preselected crystalline form of each drug and excipient nanoparticle in the aggregate particle. Advantageously, it ensures that the selected thermodynamically stable crystalline form of the drug and excipient is achieved from the aggregate particles produced by spray drying. This method of therefore advantageously controls attributes which could affect chemical and physical stability during production, storage and use of the inhalable aggregate particles.

### Example 2

The purpose of this example was to demonstrate the technique of manufacturing two-component respiratory particles comprised of nanoparticulate drug and a binder. Samples 10 through 13 were produced by bead milling drug, then adding a solution of binder to the nanosuspension prior to spray drying. Suspensions were diluted down to 5% w/v with vehicle prior to spray drying.

FIG. 5 displays typical SEM micrographs of the spray dried particles. Similarly to Example 1, the spray dried particles were spherical to irregular in shape. Table 5 lists the PSD results for the samples. Following spray drying, the two-component particles were within the respirable size range. No significant difference in PSD was observed with increasing binder concentration. Compared to the controls, the performance of samples 10 through 13 was improved.

### Example 3

The purpose of this example was to demonstrate the technique of manufacturing three-component respiratory particles comprised of nanoparticulate drug, nanoparticulate excipient and a binder. Samples 14 through 17 are illustrative cases. DPPC was used as the binder in these samples.

Samples 14, 15 and 17 used a co-milling approach in which the drug and excipient was bead milled together. A solution of DPPC binder was added to the nanosuspension mixture prior to spray drying.

Sample 16 was manufactured using an alternative approach in which the drug and excipient were bead milled separately. The nanosuspensions were then combined along with a solution of DPPC binder just before spray drying to produce the feedstock.

Suspensions were diluted down to 5% w/v with vehicle prior to spray drying.

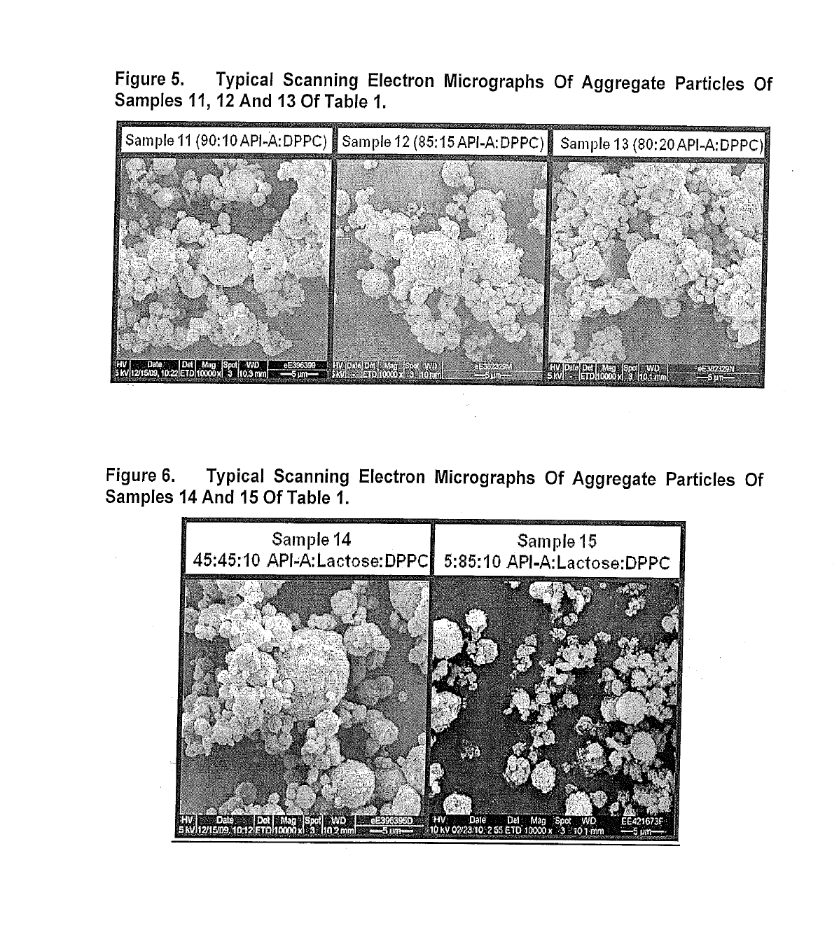

FIG. 6 displays typical SEM micrographs of the spray dried particles. Similarly to Example 1, the spray dried particles were spherical to irregular in shape. The PSD results (Table 5) for suggested the spray dried particles were within the respirable size range. The aerodynamic performance of these composite particles were improved compared to the controls. These results suggest a binder may be easily incorporated into a composite particle to improve pharmaceutical attributes.

### Example 4

The purpose of this example was to demonstrate the technique of manufacturing three-component respiratory particles comprised of nanoparticulate drug and two nanoparticulate excipients. Samples 18 through 21 utilized a co-milling approach, in which the drug and the two excipients are milled together in the bead mill to produce the feedstock suspension. Suspensions were diluted down to 5% w/v with vehicle prior to spray drying.

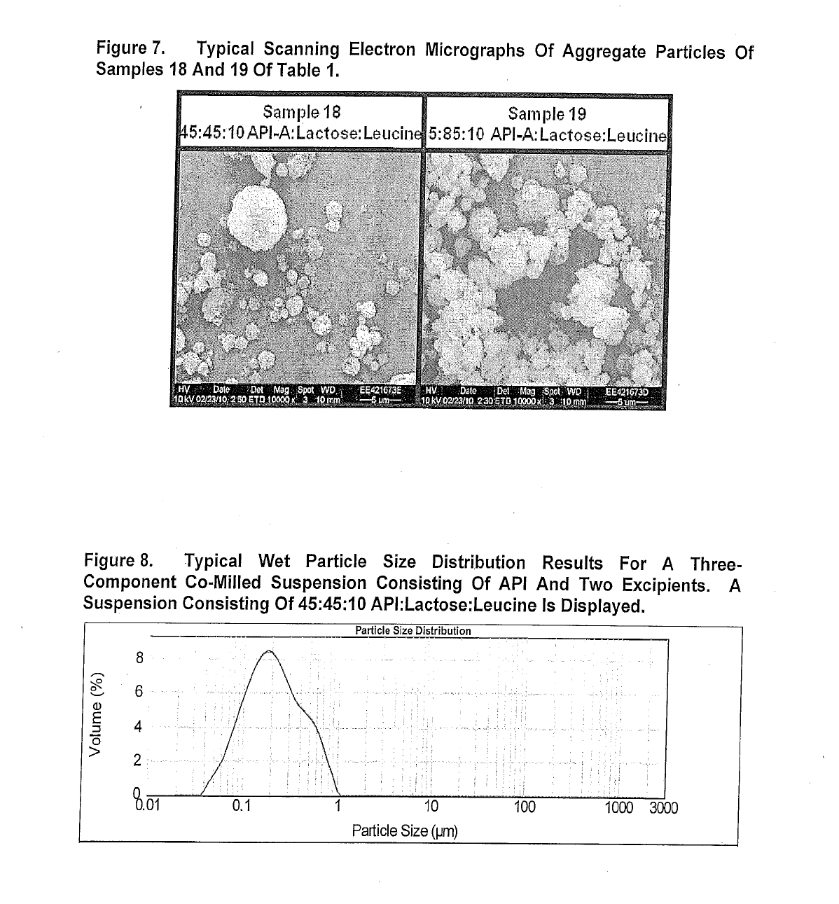

FIG. 7 displays typical SEM micrographs of the spray dried particles.

FIG. 8 illustrates typical wet PSD results obtained for three-component suspension consisting of API and two excipients. The co-milling approach produced a suspension of particles generally less than 1 micron.

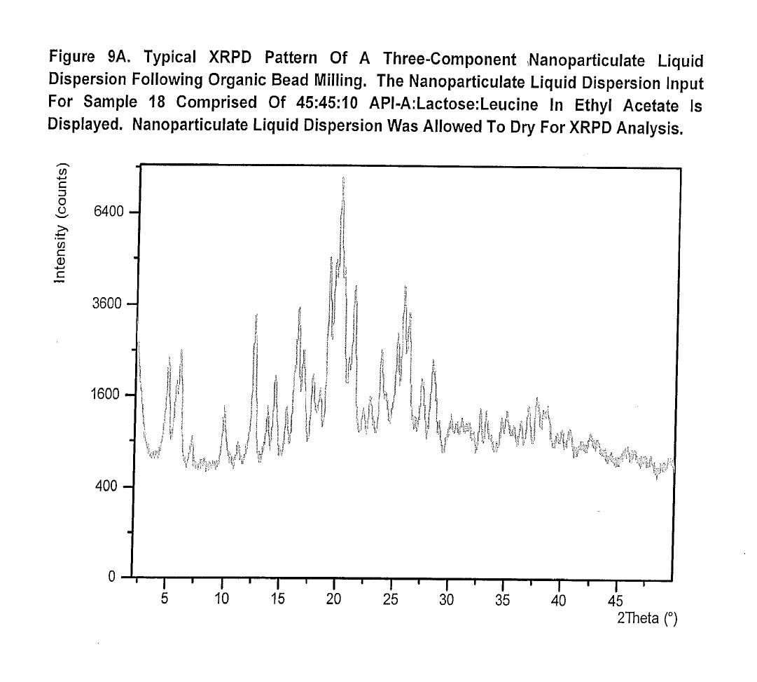

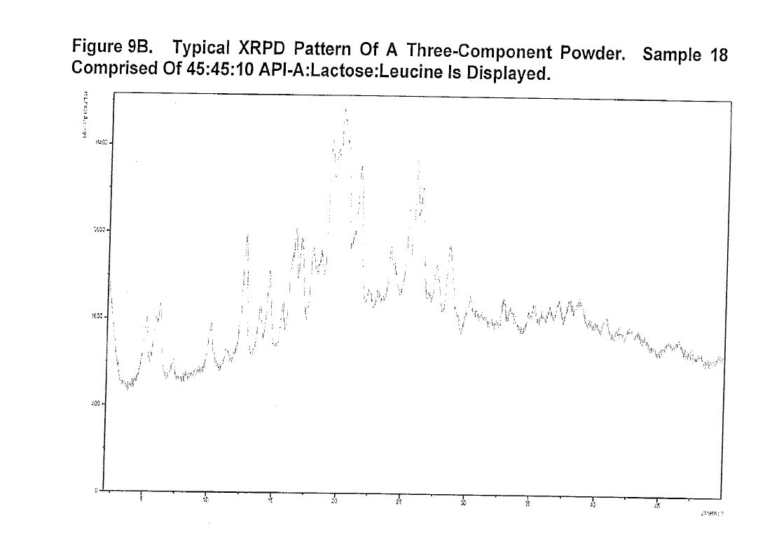

FIG. 9A shows a typical XRPD pattern for a three-component nanoparticulate liquid dispersion following non-aqueous bead milling. FIG. 9B shows a typical XRPD pattern for a three-component powder after spray drying. Similarly to Example 1, this manufacturing approach maintains the crystallinity of the input powders and produces substantially crystalline product.

Following spray drying, the PSD of these samples were within the respirable size range. Compared to the control samples, the aerodynamic performance of these samples was improved. These results illustrate how API concentration may be modified and more than one excipient flexibly incorporated into a composite particle, without sacrificing delivery efficiency.

### Example 5

The purpose of this example is to demonstrate the technique of manufacturing a blended formulation of spray dried aggregated nanoparticles and carrier excipient. Sample 5 (90:10 API-B:MgSt) was blended with coarse lactose carrier, using a suitable blender, to produce Sample 22 described in Table 5. The performance of the 10% nanoparticles composite in lactose blend was evaluated using the Diskus device and the NGI at 60 liters-per-minute.

The percent FPD of nominal was significantly greater than what is typically observed with conventional micronized API blends out of Diskus (˜25% FPD of nominal). When filled blisters were stored overwrapped and desiccated in 30° C./65% RH conditions for up to 3 months, performance was stable. These results demonstrate performance can be improved by blending spray dried aggregated nanoparticles with a carrier excipients.

TABLE 5
Physical Properties and Aerodynamic Performance of Powder Aggregate Formulations

| | | | | | | % FPD of | % FPD of |
|---|---|---|---|---|---|---|---|
| | | Mean PSD Results | | | % FPD of | Nominal (1 | Nominal (3 |
| | | X10 | X50 | X90 | Nominal | Month @30 | Month @30 |
| Sample | Formulation | (microns) | (microns) | (microns) | (Initial) | C./65% RH) | C./65% RH) |
| Control | 4% w/w mic API-A in lactose | — | — | — | 22.9 | — | — |
| Control | 100% w/w mic API-A | 0.9 | 1.9 | 3.5 | 34.8 | — | — |
| 1 | 100% w/w API-A | 0.9 | 2.0 | 4.8 | 48.4 | — | — |
| 2 | 50:50 API-A:Lactose | 0.8 | 2.3 | 7.6 | 43.4 | — | — |
| 3 | 5:95 API-A:Lactose | 0.8 | 1.8 | 3.9 | 57.1 | 61.5 | 52.8 |
| 4 | 5:95 API-A:Mannitol | 0.8 | 1.7 | 3.7 | 51.8 | 60.3 | 53.4 |
| 5 | 90:10 API-B:MgSt | 0.8 | 1.7 | 4.3 | 50.4^{a} | — | — |
| 6 | 90:10 API-A:Leucine | 0.8 | 1.9 | 4.9 | 52.1 | 53.1 | 48.7 |
| 7 | 50:50 API-A:Leucine | 0.9 | 1.8 | 4.3 | 55.1 | — | — |
| 8 | 50:50 API-A:Leucine | 0.8 | 1.9 | 4.3 | — | — | — |
| 9 | 5:95 API-A:Leucine | 0.9 | 1.9 | 3.9 | 72.2 | 53.9 | 66.2 |
| 10 | 90:10 API-A:Lecithin | 0.7 | 1.6 | 3.9 | — | — | — |
| 11 | 90:10 API-A:DPPC | 0.8 | 1.7 | 4.0 | 42.6 | 55.6 | 52.4 |
| 12 | 85:15 API-A:DPPC | 0.8 | 1.6 | 3.7 | 48.3 | — | — |
| 13 | 80:20 API-A:DPPC | 0.8 | 1.8 | 5.0 | 50.4 | — | — |
| 14 | 45:45:10 API-A:Lactose:DPPC | 0.8 | 1.9 | 7.1 | 44.1 | — | — |
| 15 | 5:85:10 API-A:Lactose:DPPC | 0.8 | 1.6 | 3.3 | 64.6 | 79.3 | 58.1 |
| 16 | 5:85:10 API-A:Lactose:DPPC | 0.7 | 1.4 | 2.9 | — | — | — |
| 17 | 5:85:10 API-A:Mannitol:DPPC | 0.7 | 1.6 | 4.9 | 44.9 | 61.1 | 43.0 |
| 18 | 45:45:10 API-A:Lactose:Leucine | 0.8 | 1.8 | 4.7 | 53.5 | 64.2 | 52.6 |
| 19 | 5:85:10 API-A:Lactose:Leucine | 0.9 | 1.9 | 3.8 | 58.8 | 59.2 | 63.2 |
| 20 | 5:85:10 API-A:Lactose:Leucine | 0.7 | 1.5 | 3.4 | 59.3 | 58.0 | 52.3 |
| 21 | 5:85:10 API-A:Mannitol:Leucine | 0.8 | 1.7 | 4.0 | 51.9 | 58.3 | 62.4 |
| 22 | 10% w/w blend of Sample 5 in | — | — | — | 51.0^{b} | 53.2^{b} | 49.1^{b} |
| | Lactose Carrier | | | | | | |

^{a}ROTAHALER ® device was used instead of CYCLOHALER ® device.
^{b}Testing performed using DISKUS ® device.

As will be appreciated from the above, controlling of the concentration of nanoparticulate drug particles and nanoparticulate excipient particles in the non-aqueous dispersion prior to aggregate formation, allows control of the concentration of drug and excipient nanoparticles ultimately making up the aggregate particles.

Potential advantages of aggregates with a low concentration of drug compared to the concentration of excipient may include the avoidance or minimization of macrophage accumulation in the lung, which is often observed in inhaled drug delivery. It is hypothesized that such aggregate particles, once deposited in the lung, readily breakdown into their nanoparticulate components, and that, further, the nanoparticles, due to their very small size may be undetectable by scavenging macrophages.

Macrophage response and macrophage accumulation, which has been observed in conventionally micronized drug blends, may be avoided with nanoparticles. With soluble nanoparticles, the enhanced dissolution rate of nanoparticles when compared to larger micron sized particles, may aid in avoiding macrophage detection. With lower solubility nanoparticles, selectively delivering active pharmaceutical agents in low local concentration may not be detectable by macrophages, again avoiding a macrophage response.

A further benefit of relatively low nanoparticulate drug particle concentration in the aggregate particles may also include a lower instance of local irritation at the deposition site of the aggregate. In this instance, a high regional lung dispersion of low drug concentration aggregate particles in a delivered aerosol dose allows less drug to be delivered per unit area of lung.

It is hypothesized that as nanoparticulate excipient particles dominate the amount of material in the aggregate particles, physical and chemical stability may be highly predictable and independent of nanoparticulate drug employed. Thus, nanoparticulate excipient particles may be employed in such aggregates to allow dilution within each aggregate particle so that dose ranging and fillability are possible.

Further, nanoparticulate drug particles and excipient particles are generated in crystalline form, thus when aggregated to form composites of respirable size, physical and chemical stability are controllable, affording benefits over conventional micronized materials, or other formation approaches.

As will be appreciated, the concentration of the nanoparticulate drug particles and nanoparticulate excipient particles in the aggregates of the present invention are controllable to achieve certain desirable ends.

Aside from the benefits otherwise described herein, the present invention potentially offers one or more significant advantages in efficiency of drug delivery as compared to conventional formulations of micronized solid drug particles. Because the aggregate particles are more aerodynamically favorable than micronized drug and excipient, a greater percentage of the dose emitted from the device actually deposits on the targeted region of the pulmonary system. For example, a typical dry powder inhaler using a micronized drug and larger excipient/carrier particles delivers as little as 10% of the metered dose to the lung. The metered dosage must be artificially increased by the manufacturer of the inhaler to assure that the therapeutically required amount of the active compound reaches the target site in the patient's body. By increasing the deposition efficiency by using particle aggregates of the present invention, this artificial increase in dosage can be reduced.

In addition, improved delivery may minimize oropharngeal deposition and reduce adverse side effects produced by drug deposition in the mouth/throat, such as oral Candidiasis (Thrush) by corticosteroids or adverse taste effects.

The patents and patent applications mentioned in this application are herein specifically incorporated by reference in their entirety.

1. A method of making aggregate particles suitable for a powder aerosol composition comprising:
(a) bead milling larger particles of drug and/or excipient in a non-aqueous liquid substantially in the absence of a homogenizing surfactant to generate nanoparticulate drug particles and/or nanoparticulate excipient particles;
(b) forming a dispersion of nanoparticulate drug particles and/or nanoparticulate excipient particles in a non-aqueous liquid,
wherein said drug particles and/or said excipient particles have a solubility of less than 10 mg/ml in said liquid dispersing media, and
wherein the nanoparticulate drug particles have a preselected crystalline form,
and wherein the non-aqueous liquid has no suspension homogenizing surfactant dissolved therein;
(c) spray-drying the dispersion of nanoparticulate drug particles and/or nanoparticulate excipient particles to generate aggregate particles comprising nanoparticulate drug particles and/or nanoparticulate excipient particles,
wherein the drug and/or excipient nanoparticles have maintained their preselected crystalline form, and
wherein the aggregate particles have a mass median aerodynamic diameter of less than or equal to about 100 microns
and wherein the aggregate particles are substantially free of a homogenizing surfactant.

2. The method of claim 1, wherein said the nanoparticles dispersed in said dispersion consist of drug.

3. The method of claim 1, wherein said the nanoparticles dispersed in said dispersion comprise drug and excipient.

4. The method of claim 1, further comprising the step of introducing a binder to the non-aqueous liquid at the spray-drying step, wherein following spray-drying said aggregate particle comprise at least one drug nanoparticle one excipient nanoparticle and binder.

5. (canceled)

6. The method of claim 1, wherein the drug and excipient are nanomilled together, simultaneously in the non-aqueous liquid dispersing media.

7-21. (canceled)

22. The method of claim 1 wherein a quantity of the drug and/or excipient is dissolved in said non-aqueous liquid.

23. (canceled)

24. The method of claim 1 wherein said non-aqueous dispersion comprises excipient, and said excipient comprises one or more amino acids, polyamino acids, stearates, sugars, synthetic sugars, sugar alcohols, or sugar acids.

25. The method of claim 24, wherein the excipient comprises leucine, magnesium stearate, lactose or mannitol, or any combination thereof.

26-28. (canceled)

29. The method of claim 1, wherein said drug comprises one or more glucocorticoid, beta agonist, anti-cholinergic, or anti-muscarinic agent, alone or in any combination.

30. (canceled)

31. The method of claim 1, wherein the non-aqueous liquid is an alcohol, ketone, ester, alkane, chlorinated hydrocarbon, fluorinated hydrocarbon, either alone or in any combination.

32. The method of claim 31, wherein said non-aqueous liquid is selected from the group consisting of ethanol, propanol, acetone, methylethylketone, ethylacetate, isopropylacetate, isooctane, cyclohexane, p11, p12, p134a, p227, and any combination thereof.

33. A powder composition suitable for delivery to the pulmonary system of a patient comprising aggregate particles prepared by the method of claim 1.

34. The powder composition of claim 33 further comprising one or more physiologically acceptable diluents or carriers.

35. A powder composition of claim 33, further comprising a physiologically acceptable coarse carrier comprising lactose, mannitol, leucine, magnesium stearate or calcium stearate, and any combination thereof.

36-37. (canceled)

38. A composition comprising aggregate particles for use in an aerosol drug delivery system, wherein the aggregate particles comprise
(a) nanoparticulate drug particles and/or
(b) nanoparticulate excipient particles, and, optionally
(c) binder,
wherein the nanoparticulate drug and/or excipient particles have a pre-selected crystalline form; and
the aggregate particles are substantially free of a homogenizing surfactant.

39. The composition of claim 38, wherein the composition includes nanoparticulate excipient particles, and said excipient particles comprise one or more amino acids, polyamino acids, sugars, synthetic sugars, sugar alcohols, sugar acids, taste masking agents, stearates, vitamin E derivatives, salts, inorganic carbonates, phosphates, cyclodextrins, EDTA di-ketopiperizines, cholesterol, cyclodextrins and inorganic phosphates, or poly(aminoacids), alone or in combination.

40. The composition of claim 39 wherein the excipient is selected from the group consisting leucine, iso-leucine, valine, glycine, trileucine; lactose, sucrose, glucose, trehalose, sucralose, mannitol, sorbitol, inositol, xylitol, erythritol, lactitol, malitol, ascorbic acid, aspartame; magnesium stearate, calcium stearate, sodium stearate; a tocopherol, a tocotrienols, sodium chloride, calcium chloride, potassium carbonate, calcium carbonate, magnesium carbonate, ammonium carbonate, sodium phosphate, potassium phosphate and calcium phosphate, a cyclodextrin, EDTA, di-keto-piperazine, and a citrate, or any combination thereof.

41-45. (canceled)

46. The composition of claim 40, wherein the nanoparticulate excipient particles are selected from the group consisting lactose, mannitol, leucine, magnesium stearate and calcium stearate, and any combination thereof.

47-71. (canceled)
